(19) United States
(12) Patent Application Publication
(10) Pub. No.: US 2006/0111287 A1
(43) Pub. Date: May 25, 2006

(54) Acetylated protein

(76) Inventor: Marco E. Bianchi, Milano (IT)

Correspondence Address: STEPTOE & JOHNSON LLP, 1330 CONNECTICUT AVENUE, N.W., WASHINGTON, DC, 20036, US

(21) Appl. No.: 10/534,254

(22) PCT Filed: Nov. 11, 2003
(86) PCT No.: PCT/IB03/05718

§ 371 Date: Aug. 31, 2005

(30) Foreign Application Priority Data
Nov. 11, 2002 (EP) 0226251.7

(51) Int. Cl. A61K 38/17; A61K 48/00; C07K 14/705; C07H 21/04; C12P 21/06

(52) U.S. Cl. 514/12; 435/69.1; 435/320.1; 435/325; 530/350; 536/23.5; 514/44

(57) ABSTRACT

An isolated multiply acetylated protein HMGB1; or a variant or fragment thereof, or a polynucleotide encoding therefor.

### FIELD OF THE INVENTION

The present invention relates to an acetylated HMGB1 protein, modulators thereof, and their use in therapy.

### BACKGROUND TO THE INVENTION

The non-histone nuclear protein HMGB1 belongs to the B family of HMG proteins, also known as the high mobility group. It has recently been reported that the non-histone nuclear protein HMGB1 is released by necrotic cells (Scaffidi et al., 2001). In living cells the protein HMGB1 does not bind to chromatin in a stable fashion; on the other hand it is sequestrated by the nuclear chromatin deacetylated during apoptosis.

EP 1 079 849 discloses the use of HMG proteins for use as the cytotoxic agent in a pharmaceutical composition. In more detail it describes administering HMG-I as a cytotoxic agent to rats having tumors. HMG-I is now designated as HMGA1, i.e. from the HMG A family, which does not have any molecular similarity which the HMG B family. No evidence is provided in EP 1079 849 in relation to the activity of the B family.

In contrast to the teaching in EP 1 079 849 our co-pending International Patent Application No. PCT/IB02/04080 (herein incorporated by reference) describes how we have now found that HMGB1 has no direct cytotoxic activity, but rather cooperates in activating immune responses. Therefore it may be used to elicit antigen-specific anti-tumor immune responses, and to complement the effect of an anti-neoplastic agent.

Extracellular protein HMGB1 determines the production of TNF-α and of other cytokines and is involved in the pathogenesis of septic shock (Anderson et al., 2000; Wang et al., 1999; WO00/47104). Moreover, the concentration of protein HMGB1 increases during haemorrhagic shock in the absence of bacterial components (Ombrellino et al., 1999). In more detail, WO00/47104 (herein incorporated by reference) describes a pharmaceutical composition for treating conditions characterised by activation of the inflammatory cytokine cascade comprising an antagonist or inhibitor of HMG1 (now designated HMGB1). WO00/47104 gives a long list of conditions which it describes as being mediated by the inflammatory cytokine cascade. In contrast to the approach of WO00/47104, our co-pending International Patent Application No. PCT/IB02/04080 describes how we have found that HMGB1 can be used to regulate an antigen mediated immune response. For example, in the approach taught in WO00/47104 conditions such as some infectious diseases and some malignancies may be treated by using an antagonist of protein HMGB1. However, following the antigen specific immune response approach we have found that administration of protein HMGB1 may be used.

Nevertheless, it will be appreciated that side effects may occur when administering HMGB1 for therapeutic purposes, e.g. for its use in treating a range of disorders associated with the acquired immune response. In particular, there may be undesirable activation of the inflammatory cytokine cascade leading to unwanted inflammation.

We have now surprisingly found that the form of protein HMGB1 which mediates the late phases of inflammation is an acetylated form of protein HMGB1. This is not taught in WO00/47104. The present invention thus provides a further method of treating conditions associated with activation of the inflammatory cytokine cascade. The present invention also provides a further method for effecting weight loss or treating obesity. The present invention provides a further method of treating a range of disorders associated with the acquired immune response preferably where side effects associated with activation of the inflammatory cytokine cascade are ameliorated.

### SUMMARY OF THE INVENTION

High Mobility Group 1 protein (HMGB1) is a chromatin component that, when leaked out by necrotic cells, triggers inflammation. Remarkably, HMGB1 can also be secreted by activated myeloid cells, and functions as a late mediator of inflammation. We show here that in all cells HMGB1 shuttles between nucleus and cytoplasm; when myeloid cells are activated, HMGB1 is acetylated on its 2 nuclear localization signals, cannot reenter the nucleus and is accumulated in secretory vesicles. Promyelocytic cells achieve HMGB1 acetylation/secretion by activating the ERK signaling pathway. We interpret this as a specific adaptation of myeloid cells to mimic the evolutionarily ancient proinflammatory signal broadcast by cells that have died by necrosis, and use it as a late inflammatory signal. However, the reinvention of HMGB1 as an actively secreted cytokine (as opposed to a passively released nuclear protein) has entailed significant post-translational modification in the form of acetylation. This post-translational modification of HMGB1 may be used to design modulators, e.g. antagonists which may be used to selectively prevent late inflammation.

The present invention also makes it possible to separate and modulate separately the “secreted cytokine” effect of protein HMGB1 from the “passively released nuclear protein” effect. There are other cases where this is desirable, for example HMGB1 is claimed in our US provisional as a chemoattractant and proliferation factor for stem cells and in W0 02/074337 as chemoattractant for smooth muscle cells.

### STATEMENTS OF THE INVENTION

According to one aspect of the present invention there is provided an isolated acetylated protein HMGB1; or a variant or fragment thereof that mimics acetylated HMGB1 (henceforth, “variant or fragment”), or a polynucleotide encoding therefor. According to another aspect there is provided an isolated acetylated HMGB1; or a variant or fragment thereof, or a polynucleotide encoding therefor, with the proviso that lysines 2 and 11 are not be acetylated. In any event, this acetylated pattern is not important for secretion by myeloid cells.

According to another aspect there is provided an isolated acetylated protein HMGB1 derivable from a myeloid cell; or a variant or fragment thereof, or a polynucleotide encoding therefor.

Preferably at least one nuclear localization signal is acetylated.

Preferably, with reference to FIG. 2C, at least one or more of lysines 27, 28, 29, 179, 181, 182, 183 or 184 are acetylated.

Preferably the protein has the acetylation pattern of FIG. 2C.

Preferably the HMGB1 is acetylated on its two nuclear localization signals.

The present invention also provides an expression vector comprising the polynucleotide of the present invention and a host cell comprising the expression vector.

According to another aspect of the present invention there is provided pharmaceutical composition comprising the acetylated protein HMGB1; or a variant or fragment thereof, or a polynucleotide encoding therefor, and a pharmaceutically acceptable carrier, excipient or diluent.

The present invention also provides a method of identifying an agent that is a modulator of acetylated protein HMGB1 or of the acetylation of protein HMGB1; or a variant or fragment thereof, or a polynucleotide encoding therefor, comprising the steps of:
(a) determining acetylated protein HMGB1 activity in the presence and absence of said agent;
(b) comparing the activities observed in step (a); and
(c) identifying said agent as a modulator by the observed differences in acetylated protein HMGB1 activity in the presence and absence of said compound.

The activity may be observed via modulation of the acetylation of protein HMGB1.

According to another aspect of the present invention there is provided a modulator of the isolated acetylated protein HMGB1 or of the acetylation of protein HMGB1; or a variant or fragment thereof.

In a preferred embodiment the modulator is specific or is to some degree selective for acetylated HMGB1 over non-acetylated HMGB1 (or HMGB1), i.e. it modulates the acetylated HMGB1 to at least some degree more than the non-acetylated form. Assays to determine the selectivity of modulators are disclosed herein. Another way to achieve some degree of selectivity is to target the acetylated pathway.

Thus the modulator may affect the activity of the acetylated protein itself or may modulate the acetylation of HMGB1, e.g. by modulating MAP (mitogen protein activated) signalling pathways, such as the ERK, p38 or Jnk signalling pathways, inhibiting active export form the nucleus, modulating the activation of myeloid cells, modulating the binding of LPS to cells, modulating the binding of inflammatory cytokines, such as IL-1β, TNF-α, LPS or HMGB1, to cell receptors, modulating the MAP kinase pathways, modulating the NF-κB pathway, modulating LPC signalling, modulating histone acetyl transferase enzymes or modulating deacetylase enzymes. Broadly we can refer to this approach as modulating the HMGB1 acetylation pathway. For example, export from the nucleus may be inhibited by using an inhibitor of CRM1/exportin binding to HMGB1, such as leptomycin B, an inhibitor of ERK's phosphorylation such as U0126, or an inhibitor of one or more histone acetyl transferase (HAT) enzymes such as pCAF, CBP and p300.

The modulator may be identifiable using the screening method of the invention.

In one embodiment the modulator is in the form of an agonist of the acetylated protein HMGB1 or a variant or fragment thereof, or a polynucleotide encoding therefor, or of the acetylation of protein HMGB1 or a variant or fragment thereof.

In another embodiment the modulator is in the form of an inhibitor of the acetylated protein HMGB1 or of the acetylation of protein HMGB1; or a variant or fragment thereof.

Preferably the inhibitor is an antibody, an antisense sequence or an acetylated protein HMGB1 receptor antagonist.

The present invention also provides a polynucleotide encoding the modulator, an expression vector comprising the polynucleotide and a host cell comprising the expression vector.

According to another aspect of the present invention there is provided a pharmaceutical composition comprising a modulator of acetylated HMGB1 and a pharmaceutically acceptable carrier, excipient or diluent.

In one embodiment the pharmaceutical composition further comprises the protein HMGB1; or a variant or fragment thereof, or a polynucleotide encoding therefor, or a modulator of the protein HMGB1 (preferably an upregulator of the protein HMGB1) or a variant or fragment thereof, or a polynucleotide encoding therefor.

In one embodiment the pharmaceutical composition is in the form of a vaccine, and may optionally further comprise an antigen and/or an APC.

According to another aspect of the present invention there is provided a method for treating a condition associated with activation of the inflammatory cytokine cascade comprising administering an effective amount of an inhibitor of acetylated HMGB1.

The condition may be sepsis or a related condition.

The method may further comprise administering a second agent in combination with the modulator, wherein the second agent is an inhibitor of an early sepsis mediator.

In one embodiment the second agent is an inhibitor of a cytokine selected from TNF, IL-1α, IL-1β, MIF and IL-6.

On another embodiment the second agent is an antibody to TNF or an IL-1 receptor antagonist (IL-1ra).

According to another aspect of the present invention there is provided the use of an inhibitor of acetylated HMGB1 for the preparation of a medicament for use in treating a condition associated with the activation of the inflammatory cascade, including sepsis or a related condition.

The present invention also provides a method of monitoring the severity and/or predicting the clinical course of sepsis and related conditions comprising measuring the concentration of acetylated protein HMGB1 in a sample, and comparing that concentration to a standard for acetylated protein HMGB1 representative of a normal concentration range of acetylated protein HMGB1 in a like sample, whereby higher levels of acetylated protein HMGB1 are indicative of severe conditions and/or toxic reactions.

The present invention further provides a method of diagnosing and/or predicting the course of conditions associated with the activation of the inflammatory cascade comprising measuring the concentration of acetylated protein HMGB1 in a sample, and comparing that concentration to a standard for acetylated protein HMGB1 representative of a normal concentration range of acetylated protein HMGB1 in a like sample, whereby higher levels of acetylated protein HMGB1 are indicative of such conditions and/or severe conditions.

In one embodiment the sample is a serum sample.

According to another embodiment of the present invention there is provided a method for effecting weight loss or treating obesity comprising administering an effective amount of acetylated protein HMGB1; or a fragment or variant thereof or a polynucleotide encoding therefor, or an upregulator of acetylated protein HMGB1 or of the acetylation of HMGB1.

According to another aspect of the present invention there is provided the use of acetylated protein HMGB1; or a fragment or variant thereof or a polynucleotide encoding therefor, or an upregulator of acetylated protein HMGB1 or of the acetylation of HMGB1 for the preparation of a medicament for use in effecting weight loss or treating obesity.

According to another aspect of the present invention there is provided use of an inhibitor of acetylated HMGB1 for administering to a patient undergoing therapy with the protein HMGB1 or a fragment or variant thereof, or a polynucleotide encoding therefor; an agonist of the protein HMGB1 or a fragment or variant thereof; or an antagonist of the protein HMGB1 or a fragment or variant thereof.

According to another aspect of the present invention there is provided the use of an inhibitor of acetylated HMGB1 for the preparation of a medicament for use in treating a patient undergoing therapy with the protein HMGB1 or a fragment or variant thereof, or a polynucleotide encoding therefor, an agonist of the protein HMGB1 or a fragment or variant thereof; or an antagonist of the protein HMGB1 or a fragment or variant thereof.

According to another aspect of the present invention there is provided a method for stimulating an immune response comprising administering the protein HMGB1 or a variant or fragment thereof, or a polynucleotide encoding therefor, and an inhibitor of acetylated HMGB1.

According to another aspect of the present invention there is provided the use of the protein HMGB1 or a variant or fragment thereof, or a polynucleotide encoding therefor, and an inhibitor of acetylated HMGB1 for the preparation of a medicament for use in stimulating an immune response.

According to another aspect of the present invention there is provided a method for the prevention of treatment of cancer or a bacterial or viral infection comprising administering the protein HMGB1 or a variant or fragment thereof, or a polynucleotide encoding therefor, and an inhibitor of acetylated HMGB1.

According to another aspect of the present invention there is provided the use of the protein HMGB1 or a variant or fragment thereof, or a polynucleotide encoding therefor, and an inhibitor of acetylated HMGB1 for the preparation of a medicament for use in treating cancer or a bacterial or viral infection.

According to another aspect of the present invention there is provided a method for producing an activated APC comprising exposing the APC to the protein HMGB1 or a variant or fragment thereof, or a polynucleotide encoding therefor, and an inhibitor of acetylated HMGB1.

In one embodiment the APC is exposed in vitro.

In another embodiment the APC is also exposed to an antigen.

In another embodiment the APC is exposed to the antigen in vivo.

In a further embodiment the inhibitor is administered in vivo.

In another embodiment the APC and/or antigen are also exposed to a T cell.

In yet another embodiment the APC and/or antigen is exposed to the T cell in vivo.

The antigen is preferably a tumor, bacterial or viral antigen.

Preferably the protein HMGB1 is in the form of a vaccine.

The present invention also provides a method of achieving tissue repair and/or regeneration; treating inflammation and facilitating and/or inducing connective tissue regeneration comprising administering the protein HMGB1, or a fragment or variant thereof, or a polynucleotide encoding therefor, and an inhibitor of acetylated HMGB1.

### DETAILED DESCRIPTION OF THE INVENTION

Various preferred features and embodiments of the present invention will now be described by way of non-limiting example.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

Modulator

As used herein, the expression “acetylation of HMGB1” is synonymous with the expression “the HMGB1 acetylation pathway” and refers to any one or more of the upstream or downstream events that result in acetylation of HMGB1.

The term “modulate” as used herein refers to a change or alteration in the biological activity of the HMGB1 acetylation pathway. Thus, modulation of HMGB1 acetylation includes inhibition or down-regulation of HMGB1 acetylation, e.g. by compounds which block, at least to some extent, the normal biological activity of the acetylation pathway. Alternatively, the term “modulation” may refer to the activation or up-regulation of HMGB1 acetylation, e.g. by compounds which stimulate or upregulate, at least to some extent, the normal biological activity of the acetylation pathway.

As used herein with regard to a biological or chemical agent, the term “modulate” includes for example enhancing or inhibiting the activity of an acetylated HMGB1 in e.g. an assay of the invention; such modulation may be direct (e.g. including, but not limited to, cleavage of—or competitive binding of another substance to the protein) or indirect (e.g. by blocking the initial production or, if required, activation of the modifying pathway).

“Modulation” refers to the capacity to either increase or decease a measurable functional property of biological activity or process by at least 10%, 15%, 20%, 25%, 50%, 100% or more; such increase or decrease may be contingent on the occurrence of a specific event, such as activation of a signal transduction pathway, and/or may be manifest only in particular cell types.

In a preferred embodiment of the present invention, the modulator of acetylated HMGB1 is used in conjunction with a modulator of HMGB1, i.e. a compound capable of up-regulating or down-regulating HMGB1.

The term “modulator” refers to a chemical compound (naturally occurring or non-naturally occurring), such as a biological macromolecule (e.g., nucleic acid, protein, non-peptide, or organic molecule), or an extract made from biological materials such as bacteria, plants, fungi, or animal (particularly mammalian) cells or tissues, or even an inorganic element or molecule. Modulators are evaluated for potential activity as inhibitors or activators (directly or indirectly) of a biological process or processes (e.g., agonist, partial antagonist, partial agonist, antagonist, inhibitors and the like) by inclusion in screening assays described herein. The activities (or activity) of a modulator may be known, unknown or partially-known. Such modulators can be screened using the methods described herein.

The term “candidate modulator” refers to a compound to be tested by one or more screening method(s) of the invention as a putative modulator. Usually, various predetermined concentrations are used for screening such as 0.01 μM, 0.1 μM, 1.0 μM, and 10.0 μM, as described more fully hereinbelow. Test compound controls can include the measurement of a signal in the absence of the test compound or comparison to a compound known to modulate the target.

The term “antagonist”, as used in the art, is generally taken to refer to a compound which binds to an enzyme and inhibits the activity of the enzyme. The term as used here, however, is intended to refer broadly to any agent which inhibits the activity of a molecule, not necessarily by binding to it. The term “antagonist” is used interchangeably with “inhibitor”.

Accordingly, it includes agents which affect the expression of a protein, or the biosynthesis of a molecule, or the expression of modulators of the activity of the inhibitor. The specific activity which is inhibited may be any activity which is characteristic of the molecule, for example, the ability to activate the late inflammation pathway.

The antagonist may bind to and compete for one or more sites on the relevant molecule, for example, the HMG box. Preferably, such binding blocks the interaction between the molecule and another entity

Blocking the activity of an acetylated HMGB1 protein or protein inhibitor may also be achieved by reducing the level of expression of the protein or inhibitor in the cell. For example, the cell may be treated with antisense compounds, for example oligonucleotides having sequences specific to the protein or protein inhibitor mRNA.

As used herein, in general, the term “antagonist” includes but is not limited to agents such as an atom or molecule, wherein a molecule may be inorganic or organic, a biological effector molecule and/or a nucleic acid encoding an agent such as a biological effector molecule, a protein, a polypeptide, a peptide, a nucleic acid, a peptide nucleic acid (PNA), a virus, a virus-like particle, a nucleotide, a ribonucleotide, a synthetic analogue of a nucleotide, a synthetic analogue of a ribonucleotide, a modified nucleotide, a modified ribonucleotide, an amino acid, an amino acid analogue, a modified amino acid, a modified amino acid analogue, a steroid, a proteoglycan, a lipid, a fatty acid and a carbohydrate. An agent may be in solution or in suspension (e.g., in crystalline, colloidal or other particulate form). The agent may be in the form of a monomer, dimer, oligomer, etc, or otherwise in a complex.

The terms “antagonist”, “upregulator” and “agent” are also intended to include, a protein, polypeptide or peptide including, but not limited to, a structural protein, an enzyme, a cytokine (such as an interferon and/or an interleukin) an antibiotic, a polyclonal or monoclonal antibody, or an effective part thereof, such as an Fv fragment, which antibody or part thereof may be natural, synthetic or humanised, a peptide hormone, a receptor, a signalling molecule or other protein; a nucleic acid, as defined below, including, but not limited to, an oligonucleotide or modified oligonucleotide, an antisense oligonucleotide or modified antisense oligonucleotide, cDNA, genomic DNA, an artificial or natural chromosome (e.g. a yeast artificial chromosome) or a part thereof, RNA, including mRNA, tRNA, rRNA or a ribozyme, or a peptide nucleic acid (PNA); a virus or virus-like particles; a nucleotide or ribonucleotide or synthetic analogue thereof, which may be modified or unmodified; an amino acid or analogue thereof, which may be modified or unmodified; a non-peptide (e.g., steroid) hormone; a proteoglycan; a lipid; or a carbohydrate. Small molecules, including inorganic and organic chemicals, which bind to and occupy the active site of the polypeptide thereby making the catalytic site inaccessible to substrate such that normal biological activity is prevented, are also included. Examples of small molecules include but are not limited to small peptides or peptide-like molecules.

HMGB1 Acetylated HMGB1 and the Acetylation Pathway

As indicated above, HMGB1 is a member of the B family of HMG proteins, also known as High Mobility Group proteins. HMGB1 is almost identical (about 99% amino acid identity) in mammals. Preferably the present invention employs human HMGB1. Rat HMGB1 is reported in Bianchi et al., 1989, Specific recognition of cruciform DNA by nuclear protein HMG1, Science 243: 1056-1059 (access No. of the sequence in the databank Y00463). Human HMGB1 and mouse HMGB1 are reported in several access numbers (for example NM_{—}002128 for human and NM_{—}010439 for mouse).

As reported in WO00/47104, HMGB1 (indicated there as HMG-1) is a 25 kDa chromosomal nucleoprotein belonging to the burgeoning high mobility group (HMG) of non-histone chromatin-associated proteins. As a group, the HMG proteins recognize unique DNA structures and have been implicated in diverse cellular functions, including determination of nucleosome structure and stability, as well as in transcription and/or replication. The HMG proteins were first characterized by Johns and Goodwin as chromatin components with a high electrophoretic mobility in polyacrylamide gels (see in The HMG Chromosomal Proteins, E. W. Johns, Academic Press, London, 1982). Higher eukaryotes exhibit three families of HMG proteins; the HMGA family (previously HMG-I/Y), the HMGB family (previously HMG1, HMG2 and HMG4), and the HMGN family (previously HMG-14/-17). The families are distinguishable by size and DNA-binding properties. HMG proteins are highly conserved across species, ubiquitously distributed and highly abundant, and are extractable from chromatin in 0.35 M NaCl and are soluble in 5% perchloric or trichloroacetic acid. Generally, HMGB proteins are thought to bend DNA and facilitate binding of various transcription factors to their cognate sequences, including for instance, progesterone receptor, estrogen receptor, HOX proteins, and Oct1, Oct2 and Oct6. Recently, it has become apparent that a large, highly diverse group of proteins including several transcription factors and other DNA-interacting proteins, contain one or more regions similar to HMGB1, and this feature has come to be known as the HMG box or HMGB domain. cDNAs coding for HMGB1 have been cloned from human, rat, mouse, mole rat, trout, hamster, pig and calf cells, and HMGB1 is believed to be abundant in all vertebrate cell nuclei. The protein is highly conserved with interspecies sequence identities in the 80% range. In chromatin, HMGB1 binds to linker DNA between nucleosomes and to a variety of non-B-DNA structures such as palindromes, cruciforms and stem-loop structures, as well as cisplatin-modified DNA. DNA binding by HMGB1 is generally believed to be sequence insensitive. HMGB1 is most frequently prepared from washed nuclei or chromatin, but the protein has also been detected in the cytoplasm. (Reviewed in Landsman and Bustin, BioEssays 15:539-546, 1993; Baxevanis and Landsman, Nucleic Acids Research 23:514-523, 1995).

In more detail, HMGB1 has a tripartite structure, composed by two homologous DNA-binding domains, the HMG-boxes, and a C-terminal domain of aspartic and glutamic acids (reviewed in Bustin, 1999, Bianchi and Beltrame, 2000, and Thomas and Travers, 2001). In most cells, HMGB1 is located in the nucleus, where it acts as an architectural protein that facilitates nucleoprotein assembly. It binds to the minor groove of DNA, inducing a local distortion of the double helix. Its lack of sequence-specificity is offset by recruitment via protein-protein interaction by different types of nuclear factors (including the Hox and Pou proteins, the steroid hormone receptors, p53, TBP, some viral proteins and the RAG1 recombinase). HMGB1 can bind to nucleosomes (Falciola et al., 1997; Nightingale et al., 1996) but in vivo its association with chromatin is very dynamic. Photobleaching experiments indicated that the average residence time of HMGB1 molecules on chromatin is less than 2 seconds (Scaffidi et al., 2002).

The phenotype of Hmgb1 knockout mice confirmed the functional importance of HMGB1 as regulator of transcription: they die shortly after birth due and show a defect in the transcriptional control exerted by the glucocorticoid receptor (Calogero et al., 1999).

Surprisingly, beyond its intranuclear function, HMGB1 also has a pivotal function outside of the cell (reviewed by Müller et al., 2001b). Wang et al. (1999a) identified HMGB1 as a late mediator of endotoxin lethality in mice, and showed that macrophages and myeloid cells stimulated by LPS, TNF or IL-1 secrete HMGB1 as a delayed response. HMGB1 can then act as a cytokine, eliciting several different responses in cells that are equipped with receptors to it. For example, HMGB1 recruits inflammatory cells and promotes the secretion of TNF. Little is known about the signaling mechanisms by which HMGB1 activates cells to respond: we showed that migration of smooth muscle cells is mediated through binding to RAGE, a multiligand receptor of the immunoglobulin superfamily that is expressed on endothelial cells, smooth muscle cells, mononuclear phagocytes and neurons (Degryse et al., 2001, and references therein).

In addition to monocytes, developing neurons and a few other cell types also secrete HMGB1 in response to specific stimuli (reviewed by Müller et al., 2001b). However, most cells are not able to secrete HMGB1 in an active manner.

In myeloid cells, secretion does not involve HMGB1 protein newly made in the cytoplasm, but proceeds through the depletion of nuclear stores. The secretion of a nuclear protein poses formidable challenges. We recently showed that activation of myeloid cells results in the redistribution of HMGB1 from the nucleus to secretory lysosomes (Gardella et al., 2002). HMGB1 does not traverse the endoplasmic reticulum and the Golgi apparatus, consistent with the absence of a leader peptide in the protein. The early mediator of inflammation interleukin (IL)-1β is also secreted by myeloid cells through a non-classical pathway involving exocytosis of secretory lysosomes (Andrei et al., 1999). However, in keeping with the roles of IL-1β and HMGB1 as early and late inflammatory factors, the discharge of the secretory vesicles that contain these 2 proteins responds at different times to different stimuli: IL-1β secretion is induced earlier by ATP, autocrinally released by myeloid cells soon after activation; HMGB1 secretion is triggered by lysophosphatidylcholine (LPC), a lipid generated later in the inflammation site (Gardella et al., 2002).

The present work provides a molecular characterization of the steps whereby HMGB1 is moved from the nucleus to secretory lysosomes. We found that in activated myeloid cells HMGB1 is extensively modified by acetylation, and that the two major clusters of acetylated lysines belong to 2 independent nuclear localization signals. We also proved that HMGB1 has non-classical nuclear export signals (NESs). Thus, in most cells HMGB1 shuttles continually from the nucleus to the cytoplasm, but the equilibrium is almost completely shifted towards a nuclear accumulation. Treatment of cells with deacetylase inhibitors causes HMGB1 acetylation, that shuts off its import into the nucleus but leaves export unaffected—the protein is then relocated to the cytoplasm. Myeloid cells acetylate HMGB1 in response to activation: in promyelocytic cells, binding of LPS or inflammatory cytokines to their surface receptors promotes the activation of the MAP kinase pathway that impinges on ERK. As a result, HMGB1 is acetylated and moves from the nucleus to the cytoplasm, where it is concentrated in secretory lysosomes and can be secreted in response to a second signal, LPC. Thus, myeloid cells have a signaling pathway that allows them to regulate HMGB1 acetylation is response to inflammatory stimuli, switching a chromatin protein into a cytokine.

HMGB1 Can Be Acetylated at Multiple Sites

We first established that HMGB1 can be multiply acetylated. The work of Allfrey and coworkers (Sterner et al., 1979) indicated that lysines 2 and 11 of HMGB1 are subject to acetylation at about the same time when histone acetylation was first discovered. This certainly holds true for most tissues and cell lines, but in thymus, in monocytes and probably in all cells of myeloid origin at least 17 different lysines within HMGB1 can be acetylated (including lysines 2 and 11), and a single HMGB1 molecule can be acetylated up to 10 times. We did not detect any other post-translational modification (other than poly-ADP-ribosylation), but in principle this level of acetylation allows for more than 100 000 different HMGB1 molecular species if all lysines were acetylated independently. In practice, several lysines appear to be acetylated concomitantly and, remarkably, the two areas of concordant lysine acetylation correspond to sequences with NLS function.

HMGB1 Shuttles Continually between Nucleus and Cytoplasm

HMGB1 (mw 25 000) is small enough to diffuse passively through nuclear pores, and in fact a significant portion of the protein diffuses to the cytoplasm if cells are incubated for a few hours at 4° C., a condition that blocks energy-driven transports. However, HMGB1 also contains two independent NLSs and two NESs, defined as CRM1-interacting surfaces in the protein. One NLS matches perfectly to classical bipartite NLSs, the other one is rather loosely related to a monopartite NLS. The 2 NESs may be related to each other as they occur in the two HMG boxes, but have no sequence similarity to other ones known to date. Two NLSs and (at least) one NES are also present in group E Sox proteins (Sox8, 9 and 10), and are essential for their transcriptional activity (Sudbeck and Scherer, 1997; Rehberg et al., 2002). Functionally, the presence of import/export signals ensures that HMGB1 shuttles actively between the nuclear and cytoplasmic compartments in all cell types, although at steady-state most HMGB1 is in the nucleus.

General deacetylase inhibitors cause the hyperacetylation of HMGB1 and the relocation of part of the protein to the cytoplasm. This presumably happens in all cell types, and may be due to the acetylation of lysines within both NLSs. The mutation of the lysine clusters within the NLSs to glutamines, which most resemble acetylated lysines, is sufficient to abrogate NLS function. The regulation of nuclear vs cytoplasmic localization by acetylation has been described before for transcription factors HNF4 and CTIIA (Soutoglou et al., 2000; Spilianakis et al., 2000); however, in these cases lysine acetylation promotes nuclear accumulation, in a process that appears to be related to NES function and protein export mechanisms. Very recently, acetylation of the NLS of viral protein E1A has been shown to cause its cytoplasmic accumulation (Madison et al., 2002). The process we describe is similar, save for the scale: around a million HMGB1 molecules per cell must be acetylated.

Myeloid Cells Control HMGB1 Acetylation

Myeloid cells and promyelocytic cells must have developed a specific ability to acetylate massively a chromatin component in order to reroute it to secretion and use it as a cytokine. Nuclear export, vesicular accumulation and secretion are separate steps, whose occurrence depends on the completion of the previous step. Thus, it is only natural that HMGB1 accumulated in secretory lysosomes should be acetylated, as this is necessary for cytoplasmic relocation of the nuclear protein. However, HMGB1 acetylation is not necessary for vesicular accumulation: secretory lysosomes do capture some of the hypoacetylated HMGB1 protein that diffuses to the cytoplasm during incubation at 4° C. of resting U937.12 cells. Most of all, secretory lysosomes also take up hypoacetylated HMGB1 released into the cytoplasm during mitosis. Thus, it appears that vesicular accumulation of cytoplasmic HMGB1 is a default process that simply requires the presence of secretory lysosomes. This further highlights the physiological importance of lysophosphatidylcholine (LPC) as a second signal for HMGB1 secretion (Gardella et al., 2002); if a second signal were not required, some secretion of HMGB1 from newly divided cells would be unavoidable.

Myeloid cells only reroute HMGB1 when activated. Activation is triggered by binding of inflammatory molecules (IL-1β, TNF-α, LPS, HMGB1 itself) to their own receptors, and is concomitant with cell differentiation. However, also non-activated U937.12 cells can transport HMGB1 to vesicles when TSA causes hyperacetylation. TSA could potentially also cause the same sort of differentiation that is triggered by inflammatory molecules, but this appears unlikely, as several morphological markers of activation are absent (not shown).

Binding of proinflammatory signals to surface receptors in myeloid cells activates a number of signaling pathways, including calcium signaling through calmodulin and NFAT/calcineurin, NF-κB and all MAP kinases (ERK, Jnk and p38 routes). We have shown that in U937.12 promyelocytic cells inhibition of ERK phosphorylation blocks the LPS-induced translocation of HMGB1 to secretory lysosomes. ERK kinases must control directly the enzymes responsible for HMGB1 acetylation, rather than indirectly through the phosphorylation of transcription factors that control the expression of specific genes, since cycloheximide treatment of either LPS-activated U937.12 cells or monocytes does not prevent HMGB1 relocation from the nucleus to cytoplasmic vesicles. Other myeloid cells (for example, monocytes, microglia, Kupffer cells, dendritic cells) may use other signalling pathways (for example the kinase pathways Jnk or p38) to control HMGB1 acetylation. Thus, translocation is controlled by ERK in U397 cells, but it may be controlled by p38 in other cells or Jnk in yet other cells, or indeed a combination of these. Indeed it has been shown that both microglial cells and macrophages respond to LPS, but one uses ERK, while the other does not (Watters et al (2002) J. Biol. Chem. 277:9077-9087; Barbour et al (1998) Mol. Immunol. 35:977-87; Rao et al (2002) J. Toxicol. Environ. Health. 65:757-68). ERK, p38 and Jnk are all serine/threonine kinases which are all downstream of ras and or/RAC/CDC42. Previously, it has been demonstrated that different myeloid cell types use different signalling pathways to control the release of a specific cytokine, for example TNF. The present invention covers all pathways used by myeloid cells that are conducive to HMGB1 acetylation.

Myeloid Cells Use the Tissue Damage Signal as a Late Inflammatory Mediator

We have recently shown that HMGB1 is passively leaked out from cells (together with all other soluble proteins) when the integrity of membranes is lost during necrosis (Degryse et al., 2001; Scaffidi et al., 2002). The release of HMGB1 by necrotic cells differs from active secretion, as it is a totally passive process: HMGB1 dilutes in the extracellular milieu following the concentration gradient. We have therefore proposed that extracellular HMGB1 is a signal for necrosis, in particular because apoptotic cells retain HMGB1 firmly bound to their chromatin even when they lose the integrity of their membranes (late apoptosis or secondary necrosis) (Scaffidi et al., 1982). Primary necrosis is caused by trauma, hypoxia or poisoning, and is associated with tissue damage to that needs repair. In this scenario the evolution of receptors like RAGE, that bind extracellular HMGB1 (Hori et al., 1995), endows cells that are not directly hurt by tissue damage with the ability to recognize, at a distance, that damage has occurred. Some cells will migrate to replace dead cells (Degryse et al., 2001), some will simply divide, and some will amplify and relay the tissue damage signal(s) to distant districts in the body. Cells of the myeloid lineage (monocytes, macrophages, neutrophils, etc.) appear to belong to this latter class: they are recruited to the site of necrosis, and are activated to secrete TNF-α and other proinflammatory cytokines (Andersson et al., 2000; Scaffidi et al., 2002). Remarkably, about 16 hours after activation, monocytes and macrophages can also secrete HMGB1 (Wang et al., 1999a), and restart a cycle of damage signaling.

The secretion of HMGB1 by cells activated by HMGB1 creates a closed feedback loop with inbuilt delay: Inflammatory cells can thus sustain the signal for tissue damage in time, and HMGB1 serves both as an early and a late inflammatory signal. This circuit is conceptually simple, economical and elegant. In our interpretation, the ability of myeloid cells to provide as output the same protein that initially served as input for inflammatory signaling is an example of molecular mimicry, and must have evolved after the evolution of HMGB1 as a tissue damage signal. The signal for tissue damage has thus evolved in general signal of danger: inflammatory cells can secrete HMGB1 also in response to TNF-α, IL-1β and LPS.

The identity of the input and output proteins for inflammation can set up a positive feedback loop, and the inflammatory response can be self-amplifying, with potentially dire consequences. The nature of the secretory process for HMGB1 provides a useful circuit breaker: the secretion of HMGB1 by monocytes takes at least 16 hours and comes later than the secretion of IL-1β, and the actual secretion (as opposed to accumulation in secretory vesicles) requires LPC as a second signal (Gardella et al., 2002). Thus, the continuation in time of the inflammatory signal is conditional.

The recognition that secreted HMGB1 is highly acetylated, whereas passively released HMGB1 is not, provides potentially the ability to inhibit only the HMGB1 that functions as late inflammation signal (for example with specific antibodies), without inhibiting the necrosis-related signaling.

Furthermore, one could target the acetylation process itself to block the secretion of HMGB1 from myeloid cells when it becomes harmful, for example during septic shock.

Variants, Fragments and Derivatives

The present invention also relates to variants, derivatives and fragments of acetylated HMGB1 and may employ variants, derivatives and fragments of HMGB1 that mimic acetylation. Preferably, the variant sequences etc. are at least as biologically active as the sequences presented herein.

As used herein “biologically active” refers to a sequence having a similar structural function (but not necessarily to the same degree), and/or similar regulatory function (but not necessarily to the same degree), and/or similar biochemical function (but not necessarily to the same degree) of the naturally occurring sequence.

Preferably such variants, derivative and fragments comprise one or both the HMG boxes.

The term “protein” includes single-chain polypeptide molecules as well as multiple-polypeptide complexes where individual constituent polypeptides are linked by covalent or non-covalent means. The term “polypeptide” includes peptides of two or more amino acids in length, typically having more than 5, 10 or 20 amino acids.

It will be understood that amino acid sequences for use in the invention are not limited to the particular sequences or fragments thereof or sequences obtained from a particular protein but also include homologous sequences obtained from any source, for example related viral/bacterial proteins, cellular homologues and synthetic peptides, as well as variants or derivatives thereof.

Thus, the present invention covers variants, homologues or derivatives of the amino acid sequences for use in the present invention, as well as variants, homologues or derivatives of the nucleotide sequence coding for the amino acid sequences used in the present invention.

In the context of the present invention, a homologous sequence is taken to include an amino acid sequence which is at least 60, 70, 80 or 90% identical, preferably at least 95 or 98% identical at the amino acid level. In particular, homology should typically be considered with respect to those regions of the sequence known to be essential for APC activation rather than non-essential neighbouring sequences. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence directly compared with the corresponding amino acid in the other sequence, one residue at a time. This is called an “ungapped” alignment Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

The terms “variant” or “derivative” in relation to the amino acid sequences of the present invention includes any substitution of, variation of, modification of, replacement of, deletion of or addition of one (or more) amino acids from or to the sequence providing the resultant amino acid sequence has APC activation activity, preferably having at least the same activity as human HMGB1.

Acetylated HMGB1 and/or HMGB1 may be modified for use in the present invention. Typically, modifications are made that maintain the activity of the sequence. Amino acid substitutions may be made, for example from 1, 2 or 3 to 10, 20 or 30 substitutions provided that the modified sequence retains the APC activation activity and/or the anti-inflammatory activity. Amino acid substitutions may include the use of non-naturally occurring analogues, for example to increase blood plasma half-life of a therapeutically administered polypeptide.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Proteins for use in the invention are typically made by recombinant means, for example as described below. However they may also be made by synthetic means using techniques well known to skilled persons such as solid phase synthesis. Proteins for use in the invention may also be produced as fusion proteins, for example to aid in extraction and purification. Examples of fusion protein partners include glutathione-S-transferase (GST), 6×His, GAL4 (DNA binding and/or transcriptional activation domains) and β-galactosidase. It may also be convenient to include a proteolytic cleavage site between the fusion protein partner and the protein sequence of interest to allow removal of fusion protein sequences. Preferably the fusion protein will not hinder the activity of the protein of interest.

Proteins for use in the invention may be in a substantially isolated form. It will be understood that the protein may be mixed with carriers or diluents which will not interfere with the intended purpose of the protein and still be regarded as substantially isolated. A protein of the invention may also be in a substantially purified form, in which case it will generally comprise the protein in a preparation in which more than 90%, e.g. 95%, 98% or 99% of the protein in the preparation is a protein of the invention.

Polynucleotides

Polynucleotides for use in the invention comprise nucleic acid sequences encoding the acetylated HMGB1 proteins, including derivatives, variants, fragments etc., and derivatives and variants which mimic acetylated HMGB1, and modulators thereof, for use in the invention. It will be understood by a skilled person that numerous different polynucleotides can encode the same protein as a result of the degeneracy of the genetic code. In addition, it is to be understood that skilled persons may, using routine techniques, make nucleotide substitutions that do not affect the protein sequence encoded by the polynucleotides of the invention to reflect the codon usage of any particular host organism in which the proteins for use in the invention are to be expressed.

Polynucleotides for use in the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides described herein may be modified by any method available in the art. Such modifications may be carried out in order to enhance the in vivo activity or life span of polynucleotides for use in the invention.

The terms “variant”, “homologue” or “derivative” in relation to the nucleotide sequence include any substitution of, variation of, modification of, replacement of, deletion of or addition of one (or more) nucleic acid from or to the sequence providing the resultant nucleotide sequence codes for a polypeptide having the capability to activate APCs.

As indicated above, with respect to sequence homology, preferably there is at least 75%, more preferably at least 85%, more preferably at least 90% homology to the sequences shown in the sequence listing herein. More preferably there is at least 95%, more preferably at least 98%, homology. Nucleotide homology comparisons may be conducted as described above. A preferred sequence comparison program is the GCG Wisconsin Bestfit program described above. The default scoring matrix has a match value of 10 for each identical nucleotide and −9 for each mismatch. The default gap creation penalty is −50 and the default gap extension penalty is −3 for each nucleotide.

The present invention also encompasses nucleotide sequences that are capable of hybridising selectively to the sequences presented herein, or any variant, fragment or derivative thereof, or to the complement of any of the above. Nucleotide sequences are preferably at least 15 nucleotides in length, more preferably at least 20, 30, 40 or 50 nucleotides in length.

The term “hybridization” as used herein shall include “the process by which a strand of nucleic acid joins with a complementary strand through base pairing” as well as the process of amplification as carried out in polymerase chain reaction technologies.

Polynucleotides for use in the invention capable of selectively hybridising to the nucleotide sequences presented herein, or to their complement, will be generally at least 70%, preferably at least 80 or 90% and more preferably at least 95% or 98% homologous to the corresponding nucleotide sequences presented herein over a region of at least 20, preferably at least 25 or 30, for instance at least 40, 60 or 100 or more contiguous nucleotides. Preferred polynucleotides for use in the invention will comprise regions homologous to the HMG box, preferably at least 80 or 90% and more preferably at least 95% homologous to the HMG box.

The term “selectively hybridizable” means that the polynucleotide used as a probe is used under conditions where a target polynucleotide for use in the invention is found to hybridize to the probe at a level significantly above background. The background hybridization may occur because of other polynucleotides present, for example, in the cDNA or genomic DNA library being screening. In this event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabelling the probe, e.g. with ³²P.

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical polynucleotide sequences while an intermediate (or low) stringency hybridization can be used to identify or detect similar or related polynucleotide sequences.

In a preferred aspect, the present invention covers nucleotide sequences that can hybridise to the nucleotide sequence of the present invention under stringent conditions (e.g. 65° C. and 0.1×SSC {1×SSC=0.15 M NaCl, 0.015 M Na₃ Citrate pH 7.0).

Where the polynucleotide for use in the invention is double-stranded, both strands of the duplex, either individually or in combination, are encompassed by the present invention. Where the polynucleotide is single-stranded, it is to be understood that the complementary sequence of that polynucleotide is also included within the scope of the present invention.

Polynucleotides which are not 100% homologous to the sequences used in the present invention but fall within the scope of the invention can be obtained in a number of ways. Other variants of the sequences described herein may be obtained for example by probing DNA libraries made from a range of individuals, for example individuals from different populations. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of the human HMGB1 sequence under conditions of medium to high stringency. Similar considerations apply to obtaining species homologues and allelic variants of the protein or nucleotide sequences for use in the invention.

Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used.

The primers used in degenerate PCR will contain one or more degenerate positions and will be used at stringency conditions lower than those used for cloning sequences with single sequence primers against known sequences.

Alternatively, such polynucleotides may be obtained by site directed mutagenesis. This may be useful where for example silent codon changes are required to sequences to optimise codon preferences for a particular host cell in which the polynucleotide sequences are being expressed. Other sequence changes may be desired in order to introduce restriction enzyme recognition sites.

Polynucleotides of the invention may be used to produce a primer, e.g. a PCR primer, a primer for an alternative amplification reaction, a probe e.g. labelled with a revealing label by conventional means using radioactive or non-radioactive labels, or the polynucleotides may be cloned into vectors. Such primers, probes and other fragments will be at least 15, preferably at least 20, for example at least 25, 30 or 40 nucleotides in length, and are also encompassed by the term polynucleotides of the invention as used herein.

Polynucleotides such as a DNA polynucleotides and probes for use in the invention may be produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art.

Longer polynucleotides will generally be produced using recombinant means, for example using a PCR (polymerase chain reaction) cloning techniques. This will involve making a pair of primers (e.g. of about 15 to 30 nucleotides) flanking a region of the lipid targeting sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector

Nucleotide Vectors

Polynucleotides of the invention can be incorporated into a recombinant replicable vector. The vector may be used to replicate the nucleic acid in a compatible host cell. Thus in a further embodiment, the invention provides a method of making polynucleotides for use in the invention by introducing a polynucleotide of the invention into a replicable vector, introducing the vector into a compatible host cell, and growing the host cell under conditions which bring about replication of the vector. The vector may be recovered from the host cell. Suitable host cells include bacteria such as E. coli, yeast, mammalian cell lines and other eukaryotic cell lines, for example insect Sf9 cells.

Preferably, a polynucleotide of the invention in a vector is operably linked to a control sequence that is capable of providing for the expression of the coding sequence by the host cell, i.e. the vector is an expression vector. The term “operably linked” means that the components described are in a relationship permitting them to function in their intended manner. A regulatory sequence “operably linked” to a coding sequence is ligated in such a way that expression of the coding sequence is achieved under condition compatible with the control sequences.

The control sequences may be modified, for example by the addition of further transcriptional regulatory elements to make the level of transcription directed by the control sequences more responsive to transcriptional modulators.

Vectors of the invention may be transformed or transfected into a suitable host cell as described below to provide for expression of a protein of the invention. This process may comprise culturing a host cell transformed with an expression vector as described above under conditions to provide for expression by the vector of a coding sequence encoding the protein, and optionally recovering the expressed protein.

The vectors may be for example, plasmid or virus vectors provided with an origin of replication, optionally a promoter for the expression of the said polynucleotide and optionally a regulator of the promoter. The vectors may contain one or more selectable marker genes, for example an ampicillin resistance gene in the case of a bacterial plasmid or a neomycin resistance gene for a mammalian vector. Vectors may be used, for example, to transfect or transform a host cell.

In preferred embodiments acetylated HMGB1 and HMGB1 may be produced by bacterial cells (Bianchi 1991, Gene 104: 271-275; Lee et al. 1998, Gene 225: 97-105), by yeasts (Mistry et al. 1997, Biotechniques 22: 718-729), or by purification from cell cultures or from mammalian tissues.

Vectors/polynucleotides for use in the invention may introduced into suitable host cells using a variety of techniques known in the art, such as transfection, transformation and electroporation. Where vectors/polynucleotides of the invention are to be administered to animals, several techniques are known in the art, for example infection with recombinant viral vectors such as retroviruses, herpes simplex viruses and adenoviruses, direct injection of nucleic acids and biolistic transformation.

Protein Expression and Purification

Host cells comprising polynucleotides of the invention may be used to express proteins for use in the invention. Host cells may be cultured under suitable conditions which allow expression of the proteins of the invention. Expression of the proteins of the invention may be constitutive such that they are continually produced, or inducible, requiring a stimulus to initiate expression. In the case of inducible expression, protein production can be initiated when required by, for example, addition of an inducer substance to the culture medium, for example dexamethasone or IPTG.

Proteins for use in the invention can be extracted from host cells by a variety of techniques known in the art, including enzymatic, chemical and/or osmotic lysis and physical disruption.

Assays

The present invention also provides a method of screening compounds to identify agonists and antagonists to acetylated HMGB1. Candidate compounds may be identified from a variety of sources, for example, cells, cell-free preparations, chemical libraries, peptide and gene libraries, and natural product mixtures. Such agonists or antagonists or inhibitors so-identified may be natural or modified substrates, ligands, receptors, enzymes, etc., as the case may be, of the retinol binding protein receptor; or may be structural or functional mimetics thereof (see Coligan et al., Current Protocols in Immunology 1(2):Chapter 5 (1991)).

The screening method may simply measure the binding of a candidate compound to acetylated HMGB1 by means of a label directly or indirectly associated with the candidate compound. Alternatively, the screening method may involve competition with a labeled competitor. Further, these screening methods may test whether the candidate compound results in a signal generated by activation or inhibition of acetylated HMGB1, using detection systems appropriate to the cells bearing the receptor. A compound which binds but does not elicit a response identifies that compound as an antagonist. An antagonist compound is also one which binds and produces an opposite response, in other words, reduction of proliferation and optionally induction of differentiation.

One assay contemplated by the invention is a two-hybrid screen. The two-hybrid system was developed in yeast [Chien et al., Proc. Natl. Acad. Sci. USA, 88: 9578-9582 (1991)] and is based on functional in vivo reconstitution of a transcription factor which activates a reporter gene. Other assays for identifying proteins that interact with acetylated HMGB1 may involve immobilizing acetylated HMGB1 or a test protein, detectably labelling the nonimmobilized binding partner, incubating the binding partners together and determining the amount of label bound. Bound label indicates that the test protein interacts with acetylated HMGB1.

Another type of assay for identifying acetylated HMGB1 interacting proteins involves immobilizing acetylated HMGB1 or a fragment thereof on a solid support coated (or impregnated with) a fluorescent agent, labelling a test protein with a compound capable of exciting the fluorescent agent, contacting the immobilized acetylated HMGB1 with the labelled test protein, detecting light emission by the fluorescent agent, and identifying interacting proteins as test proteins which result in the emission of light by the fluorescent agent. Alternatively, the putative interacting protein may be immobilized and acetylated HMGB1 may be labelled in the assay.

Also comprehended by the present invention are antibody products (e.g. monoclonal and polyclonal antibodies, single chain antibodies, chimeric antibodies, CDR-grafted antibodies and antigen-binding fragments thereof) and other binding proteins (such as those identified in the assays above) which are specific for the acetylated HMGB1 proteins of the invention. Binding proteins can be developed using isolated natural or recombinant enzymes. The binding proteins are useful, in turn, for purifying recombinant and naturally occurring enzymes and identifying cells producing such enzymes. Assays for the detection and quantification of proteins in cells and in fluids may involve a single antibody substance or multiple antibody substances in a “sandwich” assay format to determine cytological analysis of acetylated HMGB1 protein levels. The binding proteins are also manifestly useful in modulating (i.e., blocking, inhibiting, or stimulating) enzyme/substrate or enzyme/regulator interactions. Anti-idiotypic antibodies specific for mammalian checkpoint kinase binding proteins are also contemplated.

Delivery of a gene coding for a protein that mimics functional acetylated HMGB1 to appropriate cells is effected in vivo or ex vivo by use of viral vectors (e.g., adenovirus, adeno-associated virus, or a retrovirus) or ex vivo by use of physical DNA transfer methods (e.g., liposomes or chemical treatments). For reviews of gene therapy technology see Friedmann, Science, 244: 1275-1281 (1989); Verma, Scientific American: 68-84 (1990); and Miller, Nature, 357: 455-460 (1992). Alternatively, it is contemplated that in other human disease states preventing the expression of or inhibiting the activity of acetylated HMGB1 will be useful in treating the disease states. It is contemplated that antisense therapy or gene therapy could be applied to negatively regulate the expression of acetylated HMGB1. Antisense nucleic acids (preferably 10 to 20 base pair oligonucleotides) capable of specifically binding to acetylated HMGB1 expression control sequences or acetylated HMGB1 RNA are introduced into cells (e.g., by a viral vector or colloidal dispersion system such as a liposome). The antisense nucleic acid binds to the acetylated HMGB1 target sequence in the cell and prevents transcription or translation of the target sequence. Phosphothioate and methylphosphate antisense oligonucleotides are specifically contemplated for therapeutic use by the invention. The antisense oligonucleotides may be further modified by poly-L-lysine, transferrin polylysine, or cholesterol moieties at their 5′ end.

Small molecule-based therapies are particularly preferred because such molecules are more readily absorbed after oral administration and/or have fewer potential antigenic determinants than larger, protein-based pharmaceuticals. In light of the present disclosure, one of ordinary skill in the art will be able to develop drug screening methodologies which will be useful in the identification of candidate small molecule pharmaceuticals for the treatment of immune diseases. In particular, the skilled person will be able to screen large libraries of small molecules in order to identify those which bind to the normal and/or mutant/acetylated HMGB1 protein and which, therefore, are candidates for modifying the in vivo activity of the normal or mutant/acetylated proteins. Furthermore, the skilled person will be able to identify small molecules which selectively or preferentially bind to a mutant form of a acetylated HMGB1 protein.

Methods for screening small molecule libraries for candidate protein-binding molecules are well known in the art and, in light of the present disclosure, may now be employed to identify compounds which bind to the normal or mutant forms of acetylated HMGB1.

As will be obvious to one of ordinary skill in the art, there are numerous other methods of screening individual small molecules or large libraries of small molecules (e.g., phage display libraries) to identify compounds which bind to normal or mutant acetylated HMGB1. All of these methods comprise the step of mixing normal or mutant acetylated HMGB1 with test compounds, allowing for binding (if any), and assaying for bound complexes.

Compounds which bind to normal or mutant or both forms of acetylated HMGB1 may have utility in treatments. Compounds which bind only to a normal acetylated HMGB1 may, for example, act as enhancers of its normal activity and thereby at least partially compensate for the lost or abnormal activity of mutant forms of the acetylated HMGB1 in patients suffering from immune diseases. Compounds which bind to both normal and mutant forms of a acetylated HMGB1 may have utility if they differentially affect the activities of the two forms so as to alleviate the overall departure from normal function.

Once identified by the methods described above, the candidate compounds may then be produced in quantities sufficient for pharmaceutical administration or testing or may serve as “lead compounds” in the design and development of new pharmaceuticals. For example, as in well known in the art, sequential modification of small molecules (e.g., amino acid residue replacement with peptides; functional group replacement with peptide or non-peptide compounds) is a standard approach in the pharmaceutical industry for the development of new pharmaceuticals. Such development generally proceeds from a “lead compound” which is shown to have at least some of the activity of the desired pharmaceutical. In particular, when one or more compounds having at least some activity of interest are identified, structural comparison of the molecules can greatly inform the skilled practitioner by suggesting portions of the lead compounds which should be conserved and portions which may be varied in the design of new candidate compounds. Thus, the present invention also provides a means of identifying lead compounds which may be sequentially modified to produce new candidate compounds for use in the treatment of immune disease. These new compounds then may be tested both for binding (e.g., in the binding assays described above) and for therapeutic efficacy (e.g., in the animal models described herein). This procedure may be iterated until compounds having the desired therapeutic activity and/or efficacy are identified.

Compounds identified by this method will have potential utility in modifying the expression of the acetylated HMGB1 in vivo. These compounds may be further tested in the animal models disclosed and enabled herein to identify those compounds having the most potent in vivo effects. In addition, as described above with respect to small molecules having acetylated HMGB1-binding activity, these molecules may serve as “lead compounds” for the further development of pharmaceuticals by, for example, subjecting the compounds to sequential modifications, molecular modelling, and other routine procedures employed in rational drug design.

The methods and compositions of our invention rely, in some embodiments, on blocking the activity HMGB1. It is also possible in other embodiments to use agents which upregulate HMGB1. Agents which are capable of increasing the activity of HMGB1 are referred to as agonists of that activity. Similarly, antagonists reduce the activity of the HMGB1.

Antisense Compounds

As described above, the antagonist may comprise one or more antisense compounds, including antisense RNA and antisense DNA, which are capable of reducing the level of expression of the acetylated HMGB1. Preferably, the antisense compounds comprise sequences complementary to the mRNA encoding the HMGB1.

Preferably, the antisense compounds are oligomeric antisense compounds, particularly oligonucleotides. The antisense compounds preferably specifically hybridize with one or more nucleic acids encoding the HMGB1. As used herein, the term “nucleic acid encoding HMGB1 encompasses DNA encoding the HMGB1, RNA (including pre-mRNA and mRNA) transcribed from such DNA, and also cDNA derived from such RNA. The specific hybridization of an oligomeric compound with its target nucleic acid interferes with the normal function of the nucleic acid. This modulation of function of a target nucleic acid by compounds which specifically hybridize to it is generally referred to as “antisense”. The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of the HMGB1. In the context of the present invention, “modulation” means either an increase (stimulation) or a decrease (inhibition) in the expression of a gene. For example, the expression of a gene encoding an inhibitor of HMGB1 activity, or an inhibitor of expression of the HMGB1 may be increased. However, preferably, inhibition of expression, in particular, inhibition of HMGB1 expression, is the preferred form of modulation of gene expression and mRNA is a preferred target.

Antisense constructs are described in detail in U.S. Pat. No. 6,100,090 (Monia et al), and Neckers et al., 1992, Crit Rev Oncog 3(1-2):175-231.

Antibodies

The invention also provides monoclonal or polyclonal antibodies to proteins for use in the invention or fragments thereof. Thus, the present invention further provides a process for the production of monoclonal or polyclonal antibodies to proteins for use in the invention.

The acetylated HMGB1 of the present invention or derivatives or variants thereof, or cells expressing the same can be used to produce antibodies immunospecific for such polypeptides. The term “immunospecific” means that the antibodies have substantially greater affinity for the acetylated HMGB1 of the present invention than for other related polypeptides.

If polyclonal antibodies are desired, a selected mammal (e.g., mouse, rabbit, goat, horse, etc.) is immunised with an immunogenic polypeptide bearing an HMGB1 epitope(s). Serum from the immunised animal is collected and treated according to known procedures. If serum containing polyclonal antibodies to an epitope contains antibodies to other antigens, the polyclonal antibodies can be purified by immunoaffinity chromatography. Techniques for producing and processing polyclonal antisera are known in the art. In order that such antibodies may be made, the invention also provides polypeptides of the invention or fragments thereof haptenised to another polypeptide for use as immunogens in animals or humans.

Monoclonal antibodies directed against epitopes in the polypeptides of the invention can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against epitopes can be screened for various properties; i.e., for isotype and epitope affinity.

An alternative technique involves screening phage display libraries where, for example the phage express scFv fragments on the surface of their coat with a large variety of complementarity determining regions (CDRs). This technique is well known in the art.

Antibodies, both monoclonal and polyclonal, which are directed epitopes are particularly useful in diagnosis, and those which are neutralising are useful in passive immunotherapy. Monoclonal antibodies, in particular, may be used to raise anti-idiotype antibodies. Anti-idiotype antibodies are immunoglobulins which carry an “internal image” of the antigen of the agent against which protection is desired.

Techniques for raising anti-idiotype antibodies are known in the art. These anti-idiotype antibodies may also be useful in therapy.

For the purposes of this invention, the term “antibody”, unless specified to the contrary, includes fragments of whole antibodies which retain their binding activity for a target antigen. Such fragments include Fv, F(ab′) and F(ab′)₂ fragments, as well as single chain antibodies (scFv). Furthermore, the antibodies and fragments thereof may be humanised antibodies, for example as described in EP-A-239400.

Antibodies may be used in method of detecting polypeptides of the invention present in biological samples by a method which comprises:

(a) providing an antibody of the invention;

(b) incubating a biological sample with said antibody under conditions which allow for the formation of an antibody-antigen complex; and
(c) determining whether an antibody-antigen complex comprising said antibody is formed.

Suitable samples include extracts from tissues such as brain, breast, ovary, lung, colon, pancreas, testes, liver, muscle and bone tissues or from neoplastic growths derived from such tissues.

Antibodies of the invention may be bound to a solid support and/or packaged into kits in a suitable container along with suitable reagents, controls, instructions and the like.

Therapeutic Proteins

Proteins of the present invention may be administered therapeutically to patients. It is preferred to use proteins that do not consisting solely of naturally-occurring amino acids but which have been modified, for example to reduce immunogenicity, to increase circulatory half-life in the body of the patient, to enhance bioavailability and/or to enhance efficacy and/or specificity.

A number of approaches have been used to modify proteins for therapeutic application. One approach is to link the peptides or proteins to a variety of polymers, such as polyethylene glycol (PEG) and polypropylene glycol (PPG)—see for example U.S. Pat. Nos. 5,091,176, 5,214,131 and U.S. Pat. No. 5,264,209.

Replacement of naturally-occurring amino acids with a variety of uncoded or modified amino acids such as D-amino acids and N-methyl amino acids may also be used to modify proteins

Another approach is to use bifunctional crosslinkers, such as N-succinimidyl 3-(2 pyridyldithio) propionate, succinimidyl 6-[3-(2 pyridyldithio) propionamido]hexanoate, and sulfosuccinimidyl 6-[3-(2 pyridyldithio) propionamido]hexanoate (see U.S. Pat. No. 5,580,853).

It may be desirable to use derivatives of the proteins of the invention which are conformationally constrained. Conformational constraint refers to the stability and preferred conformation of the three-dimensional shape assumed by a protein. Conformational constraints include local constraints, involving restricting the conformational mobility of a single residue in a protein; regional constraints, involving restricting the conformational mobility of a group of residues, which residues may form some secondary structural unit; and global constraints, involving the entire protein structure.

The active conformation of the protein may be stabilised by a covalent modification, such as cyclization or by incorporation of gamma-lactam or other types of bridges. For example, side chains can be cyclized to the backbone so as create a L-gamma-lactam moiety on each side of the interaction site. See, generally, Hruby et al., “Applications of Synthetic Peptides,” in Synthetic Peptides: A User's Guide: 259-345 (W.H. Freeman & Co. 1992). Cyclization also can be achieved, for example, by formation of cysteine bridges, coupling of amino and carboxy terminal groups of respective terminal amino acids, or coupling of the amino group of a Lys residue or a related homolog with a carboxy group of Asp, Glu or a related homolog. Coupling of the alpha-amino group of a polypeptide with the epsilon-amino group of a lysine residue, using iodoacetic anhydride, can be also undertaken. See Wood and Wetzel, 1992, Int'l J. Peptide Protein Res. 39: 533-39.

Another approach described in U.S. Pat. No. 5,891,418 is to include a metal-ion complexing backbone in the protein structure. Typically, the preferred metal-peptide backbone is based on the requisite number of particular coordinating groups required by the coordination sphere of a given complexing metal ion. In general, most of the metal ions that may prove useful have a coordination number of four to six. The nature of the coordinating groups in the protein chain includes nitrogen atoms with amine, amide, imidazole, or guanidino functionalities; sulfur atoms of thiols or disulfides; and oxygen atoms of hydroxy, phenolic, carbonyl, or carboxyl functionalities. In addition, the protein chain or individual amino acids can be chemically altered to include a coordinating group, such as for example oxime, hydrazino, sulfhydryl, phosphate, cyano, pyridino, piperidino, or morpholino. The protein construct can be either linear or cyclic, however a linear construct is typically preferred. One example of a small linear peptide is Gly-Gly-Gly-Gly which has four nitrogens (an N₄ complexation system) in the back bone that can complex to a metal ion with a coordination number of four.

A further technique for improving the properties of therapeutic proteins is to use non-peptide peptidomimetics. A wide variety of useful techniques may be used to elucidating the precise structure of a protein. These techniques include amino acid sequencing, x-ray crystallography, mass spectroscopy, nuclear magnetic resonance spectroscopy, computer-assisted molecular modelling, peptide mapping, and combinations thereof. Structural analysis of a protein generally provides a large body of data which comprise the amino acid sequence of the protein as well as the three-dimensional positioning of its atomic components. From this information, non-peptide peptidomimetics may be designed that have the required chemical functionalities for therapeutic activity but are more stable, for example less susceptible to biological degradation. An example of this approach is provided in U.S. Pat. No. 5,811,512.

Techniques for chemically synthesising therapeutic proteins of the invention are described in the above references and also reviewed by Borgia and Fields, 2000, TibTech 18: 243-251 and described in detail in the references contained therein.

Cells

The present application has applicability in relation to any cell secretes HMGB1. Such cells include myeloid cells and neurons. Examples of myeloid cells to which the invention may be applied include promyelocytic cells, macrophages, monocytes, microglia, Kupffer cells, dentritic cells. Preferably the present invention involves promyelocytic cells or monocytes.

Therapeutic Uses

This includes any therapeutic application that can benefit a human or non-human animal. The treatment of mammals is particularly preferred. Both human and veterinary treatments are within the scope of the present invention.

Treatment may be in respect of an existing condition or it may be prophylactic. It may be of an adult, a juvenile, an infant, a foetus, or a part of any of the aforesaid (e.g. an organ, tissue, cell, or nucleic acid molecule).

Inflammatory Cytokine Cascade

In one embodiment the present invention provides a pharmaceutical composition and method for treating diseases characterized by activation of an inflammatory cytokine cascade, particularly sepsis, including septic shock and ARDS (acute respiratory distress syndrome), comprising administering an effective amount of an antagonist to acetylated HMGB1. The present invention further provides a diagnostic method for monitoring the severity of sepsis and related conditions, comprising measuring the serum concentration of HMGB1 in a patient exhibiting symptoms of a disease characterized by activation of inflammatory cytokine cascade.

Sepsis is an often fatal clinical syndrome that develops after infection or injury. Sepsis is the most frequent cause of mortality in hospitalized patients. Experimental models of gram negative sepsis based on administration of bacterial endotoxin (lipopolysaccharide, LPS) have led to an improved understanding of the pathogenic mechanisms of lethal sepsis and conditions related to sepsis by virtue of the activation of a common underlying inflammatory cytokine cascade. This cascade of host-response mediators includes TNF, IL-1, PAF and other macrophage-derived factors that have been widely studied as acute, early mediators of eventual lethality in severe endotoxemia (Zhang and Tracey, In The Cytokine Handbook, 3rd ed. Ed. Thompson (Academic Press Limited, USA). 515-547, 1998).

Unfortunately, therapeutic approaches based on inhibiting these individual “early” mediators of endotoxemia have met with only limited success in large prospective clinical trials against sepsis in human patients. It is possible to infer from these disappointing results that later-appearing factors in the host response might critically determine pathogenesis and/or lethality in sepsis and related disorders. Accordingly, there is a need to discover such putative “late” mediators necessary and/or sufficient for part or all of the extensive multisystem pathogenesis, or for the lethality, of severe endotoxemnia, particularly as endotoxemia is representative of clinical sepsis and related clinical disorders.

Diseases and conditions mediated by the inflammatory cytokine cascade are numerous. Such conditions include the following grouped in disease categories:

Systemic Inflammatory Response Syndrome, which includes:

Sepsis syndrome

Gram positive sepsis

Gram negative sepsis

Culture negative sepsis

Fungal sepsis

Neutropenic fever

Urosepsis

Meningococcemia

Trauma hemorrhage

Hums

Ionizing radiation exposure

Acute pancreatitis

Adult respiratory distress syndrome (ARDS)

Reperfusion Injury, which includes

Post-pump syndrome

Ischemia-reperfusion injury

Cardiovascular Disease, which includes

Cardiac stun syndrome

Myocardial infarction

Congestive heart failure

Infectious Disease, which includes

HIV infection/HIV neuropathy

Meningitis

Hepatitis

Septic arthritis

Peritonitis

Pneumonia Epiglottitis

E. coli 0157:H7

Hemolytic uremic syndrome/thrombolytic thrombocytopcnic purpura

Malaria

Dengue hemorrhagic fever

Leishmaniasis

Leprosy

Toxic shock syndrome

Streptococcal myositis

Gas gangrene

Mycobacterium tuberculosis

Mycobaclerium aviun intracellulare

Pyneumocystis carinii pneumonia

Pelvic inflammatory disease

Orchitis/epidydimitis

Legionella

Lyme disease

Influenza A

Epstein-Barr Virus

Viral associated hemiaphagocytic syndrome

Viral encephalitis/aseptic meningitis

Obstetrics/Gynecology, including:

Premature labor

Miscarriage

Infertility

Inflammatory Disease/Autoimmunity, which includes:

Rheumatoid arthritis/seronegative arthropathies

Osteoarthritis

Inflationary bowel disease

Systemic lupus erythematosis

Iridoeyelitis/uveitistoptic neuritis

Idiopathic pulmonary fibrosis

Systemic vasculitis/Wegener's gramilomatosis

Sarcoidosis

Orchitis/vasectomy reversal procedures

Allergic/Atopic Diseases, which includes:

Asthma

Allergic rhinitis

Eczema

Allergic contact dermatitis

Allergic conjunctivitis

Hypersensitivity pneumonitis

Malignancy, which includes:

ALL

AML

CML

CLL

Hodgkin's disease, non-Hodgkin's lymphoma

Kaposi's sarcoma

Colorectal carcinoma

Nasopharyngeal carcinoma

Malignant histiocytosis

Paraneoplastic syndrome/hypercalcemia of malignancy

Transplants, including:

Organ transplant rejection

Graft-versus-host disease

Cachexia

Congenital, which includes:

Cystic fibrosis

Familial hematophagocytic lymphohistiocytosis

Sickle cell anemia

Dermatologic, which includes:

Psoriasis

Alopecia

Neurologic, which includes:

Multiple sclerosis

Migraine headache

Renal, which includes:

Nephrotic syndrome

Hemodialysis

Uremia

Toxicity, which includes:

OKT3 therapy

Anti-CD3 therapy

Cytokine therapy

Chemotherapy

Radiation therapy

Chronic salicylate intoxication

Metabolic/Idiopathic, which includes:

Wilson's disease

Hemachromatosis

Alpha-1 antitrypsin deficiency

Diabetes

Hashimoto's thyroiditis

Osteoporosis

Hypothalamic-pituitary-adrenal axis evaluation

Primary biliary cirrhosis

Thus the present invention provides a pharmaceutical composition for treating conditions (diseases) mediated by the inflammatory cytokine cascade, comprising an effective amount of an antagonist or inhibitor of acetylated HMGB1. Preferably, the HMGB1 antagonist is selected from the group consisting of antibodies that bind to an acetylated HMGB1 protein, acetylated HMGB1 gene antisense sequences and acetylated HMGB1 receptor antagonists. The present invention provides a method for treating a condition mediated by the inflammatory cytokine cascade, comprising administering an effective amount of an acetylated HMGB1 antagonist. In another embodiment, the inventive method further comprises administering a second agent in combination with the acetylated HMGB1 antagonist, wherein the second agent is an antagonist of an early sepsis mediator, such as TNF, IL-1α, IL-1β, MIF or IL-6. Most preferably, the second agent is an antibody to TNF or an IL-1 receptor antagonist (IL-1ra).

The present invention further provides a diagnostic and prognostic method for monitoring the severity and predicting the likely clinical course of sepsis and related conditions for a patient exhibiting shock-like symptoms or at risk to exhibit symptoms associated with conditions mediated by the inflammatory cascade. The inventive diagnostic and prognostic method comprises measuring the concentration of acetylated HMGB1 in a sample, preferably a serum sample, and comparing that concentration to a standard for acetylated HMGB1 representative of a normal concentration range of acetylated HMGB1 in a like sample, whereby higher levels of acetylated HMGB1 are indicative of poor prognosis or the likelihood of toxic reactions. The diagnostic method may also be applied to other tissue or fluid compartments such as cerebrospinal fluid or urine.

The diagnostic assay provided here uses anti-acetylated HMGB1 antibodies that can be either polycolonal or monoclonal or both. The diagnostic procedure can utilize standard antibody-based techniques for measuring concentrations of the gene product of acetylated HMGB1 genes in a biological fluid. Preferred standard diagnostic procedures are ELISA assays and Western techniques.

Weight Loss/Obesity

The present invention provides a pharmaceutical composition and method for effecting weight loss or treating obesity, comprising administering an effective amount of acetylated HMGB1 or a therapeutically active fragment thereof.

Immune Response

In a preferred embodiment of the present invention an inhibitor of acetylated protein HMGB1 may be used in conjunction with HMGB1 in a method of modulating an immune response, and in particular an antigen-mediated immune response.

As described in our co-pending International patent application No. PCT/IB02/04080, in addition to triggering non-specific mechanisms, pathogens, e.g. during an infection, also trigger the antigen-specific adaptive immune response. The adaptive immune response to infection involves both the T and B cell mediated compartments of the immune system. During the so-called induction phase during antigen presenting cells (APCs) are involved in the initiation of the adaptive immune response. APC function is also required for maintenance of the adaptive immune response.

In more detail, APCs constitute a complex of cells capable of internalizing an antigen, processing it and expressing epitopes thereof in association with class I and class II MHC molecules. In general it can be said that the common characteristic of the cells of the group of APCs used medically is the expression of MHC molecules of class II as well as class I on the cell surface. The group mainly comprises dendritic cells, activated macrophages, microglial cells of the central nervous system and B lymphocytes. Among these, the dendritic cells (DCs) are particularly specialized in antigen presentation and constitute a population with distinctive characteristics and are widely distributed in tissues. The DCs are involved in the activation of the immune response, which takes place by stimulation of the T lymphocytes in the course of various pathologies such as infections, autoimmune diseases and transplant rejection. Activation or maturation of DCs is a necessary process for “priming” the T cells and initiating the immune response.

In autoimmune diseases and in transplant rejection, i.e. in the absence of pathogenic agents, induction of maturation of the dendritic cells takes place by means of endogenous molecules possessing immunostimulatory activity in vivo. It is known that cells that are dying contain and release molecules that are able to amplify the immune response (Gallucci et al., 1999; Sauter et al., 2000; Ignatius et al., 2000; Shi et al., 2000; Basu et al., 2000; Larsson et al., 2001) These molecules, normally segregated inside living cells, remain thus in the apoptotic process while they are released during cell death.

Cellular constituents released in the culture medium after cell death are able to provoke maturation of the DCs (Gallucci et al., 1999; Sauter et al., 2000). On the other hand, DCs stimulated with cells in the initial apoptotic state, or with their culture medium, are not activated (Gallucci et al., 1999; Sauter et al., 2000; Ignatius et al., 2000; Rovere et al.). Similarly, the DCs are not activated by necrotic polymorphonuclear (PMN) leukocytes.

We have found that HMGB1 is capable of activating the maturation of APCs. By “activating” we include inducing maturation of APCs. Conversely we have found that an antagonist of HMGB1 is capable of preventing or reducing the activation of an APC. Thus, for example, when an antagonist of HMGB1 is added to a population of APCs in conditions in which maturation is capable of occurring, fewer APCs proceed to maturity than in the absence of the HMGB1 antagonist.

The modulator of acetylated protein HMGB1 may be used in conjunction with this approach. In a particularly preferred embodiment, the inhibitor of acetylated protein HMGB1 may be used with an antagonist of HMGB1. This approach allows the cytokine effect of HMGB1, e.g. as an activator of APCs, to be modulated separately from acetylated HMGB1, and hence the toxic effects associated with the administration of HMGB1 due to late inflammation can be removed or reduced.

APCs

Antigen presenting cells (APCs) include macrophages, dentritic cells, B cells and virtually any other cell type capable of expressing an MHC molecule.

Macrophages are phagocytic cells of the monocytic lineage residing within tissues and are particularly well equipped for effective antigen presentation. They generally express MHC class II molecules and along with their phagocytic properties are extremely efficient at engulfing macromolecular or particulate material, digesting it, processing it with an extensive lysosomal system to antigenic peptide form, and expressing it on the cell surface for recognition by T lymphocytes.

Dendritic cells, so named for their highly branched morphology, are found in many organs throughout the body, are bone marrow-derived and usually express high levels of MHC class II antigen. Dendritic cells are actively motile and can recirculate between the bloodstream and tissues. In this way, they are considered the most important APCs. Langerhans cells are an example of dendritic cells that are located in the skin.

B lymphocytes, while not actively phagocytic, are class II-positive and possess cell surface antigen-specific receptors, immunoglobulin, or antibody molecules. Due to their potential for high affinity antigen binding, B cells are uniquely endowed with the capacity to concentrate low concentrations of antigen on their surface, endocytose it, process it and present it in the context of antigenic peptide in association with MHC antigen on their surface. In this manner, B cells become extremely effective APCs.

The APCs prepared by the method of the invention may be administered to a patient suffering from a malignancy.

Generally, in an ex vivo approach the patient will be the same patient from whom the treated APCs originated. Examples of malignancies that may be treated include cancer of the breast, cervix, colon, rectum, endometrium, kidney, lung, ovary, pancreas, prostate gland, skin, stomach, bladder, CNS, oesophagus, head-or-neck, liver, testis, thymus or thyroid. Malignancies of blood cells, bone marrow cells, B-lymphocytes, T-lymphocytes, lymphocytic progenitors or myeloid cell progenitors may also be treated.

The tumour may be a solid tumour or a non-solid tumour and may be a primary tumour or a disseminated metastatic (secondary) tumour. Non-solid tumours include myeloma; leukaemia (acute or chronic, lymphocytic or myelocytic) such as acute myeloblastic, acute promyelocytic, acute myelomonocytic, acute monocytic, erythroleukaemia; and lymphomas such as Hodgkin's, non-Hodgkin's and Burkitt's. Solid tumours include carcinoma, colon carcinoma, small cell lung carcinoma, non-small cell lung carcinoma, adenocarcinoma, melanoma, basal or squamous cell carcinoma, mesothelioma, adenocarcinoma, neuroblastoma, glioma, astrocytoma, medulloblastoma, retinoblastoma, sarcoma, osteosarcoma, rhabdomyosarcoma, fibrosarcoma, osteogenic sarcoma, hepatoma, and seminoma.

Typically the composition of the present invention may be administered with a tumour-specific antigen such as antigens which are overexpressed on the surface of tumour cells.

The APCs may be used to treat an ongoing immune response (such as an allergic condition or an autoimmune disease) or may be used to generate tolerance in a patient. Thus the cells of the present invention may be used in therapeutic methods for both treating and preventing diseases characterised by inappropriate lymphocyte activity in animals and humans. The APCs may be used to confer tolerance to a single antigen or to multiple antigens.

Typically, APCs are obtained from the patient or donor and primed as described above before being returned to the patient (ex vivo therapy).

Particular conditions that may be treated or prevented include multiple sclerosis, rheumatoid arthritis, diabetes, allergies, asthma, and graft rejection. The present invention may also be used in organ transplantation or bone marrow transplantation.

Vaccine

Another aspect of the invention relates to a method for inducing an immunological response in an individual, particularly a mammal, preferably humans, which comprises inoculating the individual with the HMGB1 protein of the present invention, or a fragment or variant thereof, adequate to produce antibody and/or T cell immune response to protect said individual from for example a tumor or infection such as a bacterial or viral infection. Also provided are methods whereby such immunological response slows tumor growth or viral or bacterial replication.

A further aspect of the invention relates to an immunological composition that when introduced into an individual, preferably a human, capable of having induced within it an immunological response, induces an immunological response in such individual. The immunological response may be used therapeutically or prophylactically and may take the form of antibody immunity and/or cellular immunity, such as cellular immunity arising from CTL or CD4+ T cells.

The immunological response may be to a HMGB1 protein of the present invention; however we have surprisingly found that the HMGB1 protein may be used as an adjuvant in a composition wherein the immunological response is directed to another antigen. Thus, HMGB1 may be used as an adjuvant in a vaccine composition.

The preparation of vaccines which contain an immunogenic polypeptide(s) as active ingredient(s), is known to one skilled in the art. Typically, such vaccines are prepared as injectables, either as liquid solutions or suspensions; solid forms suitable for solution in, or suspension in, liquid prior to injection may also be prepared. The preparation may also be emulsified, or the protein encapsulated in liposomes. The active immunogenic ingredients are often mixed with excipients which are pharmaceutically acceptable and compatible with the active ingredient. Suitable excipients are, for example, water, saline, dextrose, glycerol, ethanol, or the like and combinations thereof.

In addition, if desired, the vaccine may contain minor amounts of auxiliary substances such as wetting or emulsifying agents, pH buffering agents, and/or adjuvants which enhance the effectiveness of the vaccine.

The vaccine formulation of the invention preferably relates to and/or includes an adjuvant system for enhancing the immunogenicity of the formulation. Preferably the adjuvant system raises predominantly a TH1 type of response.

An immune response may be broadly distinguished into two extreme categories, being a humoral or cell mediated immune responses (traditionally characterised by antibody and cellular effector mechanisms of protection respectively). These categories of response have been termed TH1-type responses (particularly efficient against intracellular pathogens and tumor cells), and TH2-type immune responses (humoral response, mainly involved in the response to extracellular pathogens).

Extreme TH1-type immune responses may be characterised by the generation of antigen specific, haplotype restricted cytotoxic T lymphocytes, and natural killer cell responses. In mice TH1-type responses are often characterised by the generation of antibodies of the IgG2a subtype, whilst in the human these correspond to IgG1 type antibodies. TH2-type immune responses are characterised by the generation of a broad range of immunoglobulin isotypes including in mice IgG1, IgA, and IgM.

It can be considered that the driving force behind the development of these two types of immune responses are cytokines. High levels of TH1-type cytokines tend to favour the induction of cell mediated immune responses to the given antigen, whilst high levels of TH2-type cytokines tend to favour the induction of humoral immune responses to the antigen.

The distinction of TH1 and TH2-type immune responses is not absolute. In reality an individual will support an immune response which is described as being predominantly TH1 or predominantly TH2. However, it is often convenient to consider the families of cytokines in terms of that described in murine CD4+ve T cell clones by Mosmann and Coffman (Mosmann, T. R. and Coffman, R. L. (1989) TH1 and TH2 cells: different patterns of lymphokine secretion lead to different functional properties. Annual Review of Immunology, 7, p145-173). Traditionally, TH1-type responses are associated with the production of the INF-γ and IL-2 cytokines by T-lymphocytes. Other cytokines often directly associated with the induction of TH1-type immune responses are not produced by T-cells, such as IL-12. In contrast, TH2-type responses are associated with the secretion of IL-4, IL-5, IL-6 and IL-13.

It is known that certain vaccine adjuvants are particularly suited to the stimulation of either TH1 or TH2-type cytokine responses. Traditionally the best indicators of the TH1:TH2 balance of the immune response after a vaccination or infection includes direct measurement of the production of TH1 or TH2 cytokines by T lymphocytes in vitro after restimulation with antigen, and/or the measurement of the IgG1:IgG2a ratio of antigen specific antibody responses.

Thus, a TH1-type adjuvant is one which preferentially stimulates isolated T-cell populations to produce high levels of TH1-type cytokines when re-stimulated with antigen in vitro, and promotes development of both CD8+ cytotoxic T lymphocytes and antigen specific immunoglobulin responses associated with TH1-type isotype.

The HMGB1 protein may be used as an adjuvant in a variety of vaccine types. Non-limiting examples of such vaccine types include subunit vaccines and cellular vaccines, e.g. immunotherapy of tumors with dendritic cells.

The composition of the present invention may include (additional) adjuvants. Examples of adjuvants and other agents include aluminum hydroxide, aluminum phosphate, aluminum potassium sulfate (alum), beryllium sulfate, silica, kaolin, carbon, water-in-oil emulsions, oil-in-water emulsions, muramyl dipeptide, bacterial endotoxin, lipid X, Corynebacterium parvum (Propionobacterium acnes), Bordetella pertussis, polyribonucleotides, sodium alginate, lanolin, lysolecithin, vitamin A, saponin, liposomes, levamisole, DEAE-dextran, blocked copolymers or other synthetic adjuvants. Such adjuvants are available commercially from various sources, for example, Merck Adjuvant 65 (Merck and Company, Inc., Rahway, N.J.) or Freund's Incomplete Adjuvant and Complete Adjuvant (Difco Laboratories, Detroit, Mich.).

Typically, adjuvants such as Amphigen (oil-in-water), Alhydrogel (aluminum hydroxide), or a mixture of Amphigen and Alhydrogel are used. Only aluminum hydroxide is approved for human use.

The proportion of immunogen and adjuvant can be varied over a broad range so long as both are present in effective amounts. For example, aluminum hydroxide can be present in an amount of about 0.5% of the vaccine mixture (Al₂O₃ basis). Conveniently, the vaccines are formulated to contain a final concentration of immunogen in the range of from 0.2 to 200 μg/ml, preferably 5 to 50 μg/ml, most preferably 15 μg/ml.

After formulation, the vaccine may be incorporated into a sterile container which is then sealed and stored at a low temperature, for example 4° C., or it may be freeze-dried. Lyophilisation permits long-term storage in a stabilised form.

The effectiveness of an adjuvant may be determined by measuring the amount of antibodies or T cells directed against an immunogenic polypeptide containing an antigenic sequence resulting from administration of this polypeptide in vaccines which are also comprised of the various adjuvants.

The vaccines are conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkylene glycols or triglycerides; such suppositories may be formed from mixtures containing the active ingredient in the range of 0.5% to 10%, preferably 1% to 2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% to 95% of active ingredient, preferably 25% to 70%. Where the vaccine composition is lyophilised, the lyophilised material may be reconstituted prior to administration, e.g. as a suspension. Reconstitution is preferably effected in buffer

Capsules, tablets and pills for oral administration to a patient may be provided with an enteric coating comprising, for example, Eudragit “S”, Eudragit “L”, cellulose acetate, cellulose acetate phthalate or hydroxypropylmethyl cellulose.

The polypeptides of may be formulated into the vaccine as neutral or salt forms. Pharmaceutically acceptable salts include the acid addition salts (formed with free amino groups of the peptide) and which are formed with inorganic acids such as, for example, hydrochloric or phosphoric acids, or such organic acids such as acetic, oxalic, tartaric and maleic. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine and procaine.

Additional vaccinations have been recently developed that rely on the injection of APCs either subcutaneously, intradermis, intravenously, intranodally, or intra-tumour. Cells are usually resuspended in appropriate isotonic media before injection, which may be further supplemented by adjuvants.

Methods

Immature dendritic cells for use in the present invention can be obtained from haematopoietic precursors or from stem cells, for example from PBMC cells, by suitable treatment with cytokines such as GM-CSF, IL-4 and flt3-L.

The activation or maturation of antigen-presenting cells can be effected starting from a culture of immature or inactive cells, by adding HMGB1 protein and possibly other co-adjuvants such as cytokines to the culture medium.

Once led to maturation or activated, the antigen-presenting cells, especially the DCs, can be used for the activation of T lymphocytes in response to particular antigens; the lymphocytes thus activated can then be administered to a subject to stimulate their immune response to the said antigens.

The indicators of activation can vary according to the cell type under consideration. With regard to macrophages, microglia and B lymphocytes, for example, it is a functional activation with increase in membrane expression of MHC molecules and co-stimulatory molecules following contact with other adjuvants, as described in Rovere et al, 2000 and Aderem et al., 2000.

In the case of the dendritic cells, those cells that display increased expression of markers characteristic of the “maturation phenotype”, such as the CD83 and CD86 surface molecules, or reduced expression of markers characteristic of the immature phenotype, such as CD115, CD14, CD68 and CD32, are regarded as activated or mature.

According to a one embodiment, the invention therefore relates to an ex vivo method for the activation of T lymphocytes that comprises the following steps:
a) bringing a preparation of inactive APCs into contact with HMGB1, or with its biologically active fragments, so as to induce their activation;
b) bringing the activated APCs into contact with a particular antigen;
c) exposing the T lymphocytes to the APCs that have been activated and exposed to the antigen.

According to a preferred embodiment, dendritic cells are used as APCs.

Steps a)-c) indicated above can be executed in a different order. For example, the antigen can be added to a culture of immature or inactive APCs before the HMGB1 protein or its fragments. In addition, the APCs or DCs can be transfected with a vector for the expression of a particular antigen or of a polypeptide derived from it, or alternatively a vector for the expression of a specific MHC molecule. Antigens associated with microorganisms, viruses, tumours or autoimmune diseases can be used for the activation of lymphocytes according to the method described. As tumour antigens, in addition to the proteins or their fragments isolated from tumour tissues or cells, it is possible to use whole cells that have been killed by apoptosis or necrosis. It is also possible to use antigens associated with viruses or retroviruses, especially HIV, or with intracellular pathogens, such as mycobacteria or plasmodia.

In another embodiment the present invention relates to an in vivo method in which HMGB1 and optionally an antigen are introduced into a patient, for example into a lymph node or into a tumour. The antigen may be introduced before, at the same time as, or after the HMGB1. Alternatively the antigen may be present in vivo, for example as an HLA antigen or have been introduced during a transplant.

In all embodiments the HMGB1 and/or antigen may be introduced as a polynucleotide sequence, i.e. using a gene delivery approach.

Introduction of Nucleic Acid Sequences into APCs

APCs as described above may be cultured in a suitable culture medium such as DMEM or other defined media, optionally in the presence of fetal calf serum.

HMGB1 may be administered to APCs and by introducing nucleic acid constructs/viral vectors encoding the protein into cells under conditions that allow for expression of the polypeptide in the APC. Similarly, nucleic acid constructs encoding antisense constructs may be introduced into the APCs and by transfection, viral infection or viral transduction.

The antagonist of the present invention may also be administered in a similar way to HMGB1.

Stem Cell Chemoattractant and Proliferation Promoter

Our copending U.S. provisional describes the use of HMGB1 as a stem cell chemoattractant and proliferation promoter in the treatment of the first step of inflammation and of tissue repair. As discussed above we have now found that the form of HMGB1 which is secreted by myeloid cells and which mediates the late phases of inflammation is an acetylated form of HMGB1. In contrast, HMGB1 released during cellular necrosis is not necessarily acetylated, and has chemoattractant and mitogenic activity. Thus, the present invention contemplates a method to induce stem cell migration and/or proliferation in cell culture or in vivo comprising the step of exposing such cells to effective amounts of HMGB1 while blocking the concurrent inflammation by using antagonists of the acetylated HMGB1.

The present invention also refers to inhibitors of the non-acetylated form of HMGB1 and their use in cases of large scale necrosis such as intestinal infarction, acute pancreatitis and extensive trauma.

In the above cases the non-acetylated HMGB1 or inhibitor thereof may be administered with acetylated HMGB1 or an inhibitor thereof. In accordance with the present invention any reference to administration with another compound does not necessarily imply that the two compounds are administered at the same time. Instead one compound could be administered before or after the other compound.

The above methods thus avoid conventional side effects associated with inflammation.

Vascular Disease

WO02/074337 relates to the use of inhibitors of HMGB1 for the treatment of vascular disease. Such inhibitors should be directed to the non-acetylated form of HMGB1. The vascular disease includes atherosclerosis and/or restenosis that occur during angioplasty.

WO02/074337 also teaches the use of HMGB1 to facilitate and/or induce connective tissue regeneration. Again, such inhibitors should be directed to the non-acetylated form of HMGB1.

In the above cases the non-acetylated HMGB1 or inhibitor thereof may be administered with acetylated HMGB1 or an inhibitor thereof.

The above methods thus avoid conventional side effects associated with inflammation.

Delivery Systems

The invention further provides a delivery system for a protein, polynucelotide, agonist or antagonist of the present invention. For ease of reference to protein, agonist and/or antagonist will be referred to as “agent” in the present section.

The delivery system of the present invention may be a viral or non-viral delivery system. Non-viral delivery mechanisms include but are not limited to lipid mediated transfection, liposomes, immunoliposomes, lipofectin, cationic facial amphiphiles (CFAs) and combinations thereof. As previously indicated when the agent is delivered in the form of a polynucleotide to a cell for subsequent expression therein the agent is preferably delivered via a retroviral vector delivery system. However, the polynucleotide may be delivered to the target cell population by any suitable Gene Delivery Vehicle, GDV. This includes but is not restricted to, DNA, formulated in lipid or protein complexes or administered as naked DNA via injection or biolistic delivery, and viruses such as retroviruses. Alternatively, the polynucleotides are delivered by cells such as monocytes, macrophages, lymphocytes or hematopoietic stem cells. In particular a cell-dependent delivery system is used. In this system the polynucleotides encoding the agent are introduced into one or more cells ex vivo and then introduced into the patient.

The agents of the present invention may be administered alone but will generally be administered as a pharmaceutical composition.

Pharmaceutical Compositions

A pharmaceutical composition is a composition that comprises or consists of a therapeutically effective amount of a pharmaceutically active agent. It preferably includes a pharmaceutically acceptable carrier, diluent or excipients (including combinations thereof). Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

“Therapeutically effective amount” refers to the amount of the therapeutic agent which is effective to achieve its intended purpose. While individual patient needs may vary, determination of optimal ranges for effective amounts of HMGB1 is within the skill of the art. Generally the dosage regimen for treating a condition with the compounds and/or compositions of this invention is selected in accordance with a variety of factors, including the type, age, weight, sex, diet and medical condition of the patient, the severity of the dysfunction, the route of administration, pharmacological considerations such as the activity, efficacy, pharmacokinetic and toxicology profiles of the particular compound used, whether a drug delivery system is used, and whether the compound is administered as part of a drug combination and can be adjusted by one skilled in the art. Thus, the dosage regimen actually employed may vary widely and therefore may deviate from the preferred dosage regimen set forth herein.

Examples of pharmaceutically acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

Where appropriate, the pharmaceutical compositions can be administered by any one or more of: inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intracavernosally, intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Typically, each conjugate may be administered at a dose of from 0.01 to 30 mg/kg body weight, preferably from 0.1 to 10 mg/kg, more preferably from 0.1 to 1 mg/kg body weight.

When the polynucleotides/vectors are administered as a naked nucleic acid, the amount of nucleic acid administered may typically be in the range of from 1 μg to 10 mg, preferably from 100 μg to 1 mg.

Uptake of naked nucleic acid constructs by mammalian cells is enhanced by several known transfection techniques for example those including the use of transfection agents. Example of these agents include cationic agents (for example calcium phosphate and DEAE-dextran) and lipofectants (for example lipofectam™ and transfectam™). Typically, nucleic acid constructs are mixed with the transfection agent to produce a composition.

The routes of administration and dosages described are intended only as a guide since a skilled practitioner will be able to determine readily the optimum route of administration and dosage for any particular patient and condition.

### DESCRIPTION OF THE FIGURES

The present invention will now be described further with reference to the following non-limiting Examples and Figures in which:

FIG. 1. Two-dimensional Electrophoresis Reveals Many Isoforms of HMGB1

(A) HMGB1 purified from calf thymus gives rise on a mono-dimensional gel (SDS-PAGE, Coomassie stain, right panel) to two bands (arrows): a major one at about 29 kDa apparent molecular weight, plus a minor one that contains ADP-ribosylated protein. Fifty μg of the same sample of HMGB1 were subjected to 2D gel electrophoresis (silver stain, left panel); 5 μl of 2D Protein Marker from BioRad were loaded together with HMGB1, generating a matrix of spots at the molecular weights listed on the right.

(B) Total mouse thymus contains many isoforms of HMGB1. About 300 μg of protein from a thymus total extract (from a mouse embryo of 17 days) were loaded on two twin 2-D gels; the gel shown on the left was silver stained, while the other one was electroblotted onto a nitrocellulose filter and assayed with a specific anti-HMGB1 rabbit antibody. Note the similarity of the pattern to that obtained from purified HMGB1 (A).

(C) Most cell lines contain 2 isoforms of HMGB1, and the protein can be hyperacetylated with TSA treatment. 3T3 cells were incubated for 6 hours in DMEM, or DMEM supplemented with 10 ng/ml TSA, then collected and lysed. About 400 μg of whole cell extracts was loaded in duplicate on two 2D gels and then blotted on nitrocellulose; patterns of HMGB1 were revealed with polyclonal anti-HMGB1 antibody. Upon inhibition of HDACs (right), several HMGB1 spots appeared, compared to only 2 in control fibroblasts (left).

FIG. 2. Strategy for 2D/MALDI-MS Analysis of Multiply Modified HMGB1 Spots were excised from 2D gels and proteolyzed; the complex mixture of oligopeptides was analyzed by MALDI-TOF. A mass was attributed to each peptide in the mixture; the masses corresponding to peptides predicted in silico were selected as “anchors”, and we searched the complex spectra for further mass peaks corresponding to anchor masses plus multiples of 42 (the mw of an acetyl group). Two examples are given in the figure; the procedure was iterated for all peptides. We also mined the spectra for evidence of phosphorylation, methylation and glycosylation, without finding any. This information was used to infer the modification pattern on HMGB1.

(B) An example of the multi-step digestion strategy developed to determine acetylation sites in HMGB1: spots from 2D gels were digested in gel with protease Asp-N. One aliquot of the peptides thus obtained was analyzed by MALDI-TOF: the arrows identify the peaks corresponding to unmodified fragments highlighted in the sequence. Peaks corresponding to the mw of unmodified fragments plus multiples of 42 were then identified. The maximum number of acetyl moieties on each fragment was thus determined. Another aliquot of Asp-N digested HMGB1 was further digested with CNBr, and the products were analyzed similarly. We proceeded with further cleavages until we could obtain identifiable fragments where all lysines were acetylated, or none was.

(C) Final attribution of acetylation sites on the HMGB1 sequence: lysines marked with an arrow (8 out of 43) are frequently modified; lysines marked with an asterix are never modified (20/43); lysines marked with a cross (9/43) are modified with a low but detectable frequency; lysines marked with a bullet (6/43) are uncharacterized, because they are located in areas of HMGB1 with no sequence coverage (peptides could not be found in spectra). HMG boxes and the acidic tail are boxed.

FIG. 3. Identification of 2 NLSs in HMGB1

(A) Amino acids 27-43 of HMGB1 were compared with previously characterized bipartite NLSs: this sequence matches perfectly with the NLSs of human p53, progesterone receptor and Xenopus nucleoplasmin. The presumptive bipartite NLS of HMGB1 was fused to GFP and expressed in HeLa cells: while the NLS-GFP fusion is predominantly nuclear, GFP alone is broadly diffuse. Lysines (K) 27, 28, and 29 of HMGB1-GFP were changed into glutamines (O) or alanines (A): neither mutation altered the nuclear localization of the fluorescent protein.

(B) A second NLS in HMGB1. Sequence segments of HMGB1 were launched into the PredictNLS database: the 178-184 segment matched with 19 proteins in the database, 17 of them nuclear proteins. This constitutes a good indication for a potential NLS, that we named NLS2. The presumptive NLS2 was fused to GFP and expressed in HeLa cells, showing a predominantly nuclear distribution. Lysines 181, 182 and 183 of HMGB1-GFP were mutated into glutamines (O) or alanines (A): neither mutation caused the cytoplasmic localization of HMGB1-GFP.

(C) Inactivation of both NLSs causes redistribution of HMGB1-GFP to the cytoplasm. In HeLa cells, mutations of the 6 lysines in the 2 NLSs into glutamines (as a mimic of acetyllysine, upper panels) or alanines (central panels) produce a clear cytoplasmic fluorescence of comparable intensity. As negative control, a mutant in which the 6 lysines are mutated to arginines did not alter the nuclear localization of HMGB1-GFP (lower panels). Bar represents 10 μm in all panels.

FIG. 4. HMGB1 Migrates from Nucleus to Cytoplasm by Both Passive and Active Transport

(A) Heterokaryons were formed by fusing HMGB1-expressing HeLa cells (human) and Hmgb1−/− mouse embryonic fibroblasts. Human cytokeratin and HMGB1 were stained red and green, respectively, with specific antibodies; nuclei were stained blue with DAPI (or Hoechst). Cells with human cytokeratin staining and two nuclei, one of which with bright DAPI-positive heterochromatic spots characteristic of mouse cells, were considered heterokaryons. After incubation at 37° C. for 4 hours, HMGB1 re-equilibrated from human to mouse nuclei, indicating that it could pass from the nuclear to the cytoplasmic side of the nuclear membranes, and be reuptaken by the other nucleus.

(B) Leptomycin B (150 nM) substantially reduces, but does not abolish, HMGB1 transfer between human and mouse nuclei. Heterokaryons were produced and scored as in panel A; 150 heterokaryons were evaluated for HMGB1 transfer into recipient mouse nuclei for each of the 2 classes (leptomycin and control). The 50% level is equivalent to complete equilibration.

(C) Both HMG boxes interact directly with CRM1 exportin. Labeled CRM1 protein was mixed with beads bearing GST-NS2 immobilized onto Glutathione Sepharose or BSA, tailless HMGB1 (boxA+B) or individual boxes covalently linked to Sepharose. Aliquots representing the input material (In), the fourth wash (W4), the material remaining bound to beads after 5 washes (bound, B), and the unbound material (output, O) were electrophoresed and autoradiographed. CRM1 binds to all beads save the negative control BSA. Inclusion of 0.4 μM leptomicin B in the binding buffer prevents binding of CRM1 to both GST-NS2 and the NESs contained in the HMG boxes (lanes 18-23).

FIG. 5. LPS-activated Human Monocytes Hyperacetylate HMGB1 and Accumulate It in Cytoplasmic Vesicles

Primary monocytes purified from peripheral blood cells were cultured overnight, in the presence or absence of 100 ng/ml LPS. Aliquots of activated and control monocytes were then fixed and immunostained with anti-HMGB1 rabbit antibody. Note the exclusively nuclear localization of HMGB1 in unstimulated monocytes, as opposed to nuclear plus vesicular HMGB1 localization in LPS-activated monocytes. Bar represents 7 μm. Further aliquots of untreated and LPS-activated monocytes were lysed by freeze-thawing, and about 400 μg of total protein extract were loaded on 2D gels, blotted onto nitrocellulose filters, and immunodetected with anti-HMGB1. Note the major additional HMGB1 spot in activated monocytes.

FIG. 6. HMGB1 Relocates Following LPS or TSA Treatment

Exposure of 3134 fibroblasts to 10 ng/ml TSA for 3 hours causes a significant relocation of HMGB1-GFP to the cytoplasm; no vesicles are recognizable. U937.12 cells were cultured for 3 hours without stimulation, in the presence of 100 ng/ml LPS, or with 10 ng/ml TSA. Cells were then fixed and immunostained with anti-HMGB1 antibody. HMGB1 is exclusively nuclear in resting U937.12 cells, whereas it is predominantly vesicular in LPS-activated cells, that display a much larger cytoplasm. TSA treated U937.12 cells do not show cytoplasmic expansion, but a fraction of HMGB1 is relocalized to cytoplasmic vesicles. A significant fraction of HMGB1 is contained in vesicles in resting monocytic cells that have been incubated 3 hours at 4° C. to promote passive diffusion of hypoacetylated HMGB1 to the cytoplasm and rewarmed to 37° C. for 10 minutes to resume active transport. Likewise, a significant fraction of HMGB1 is contained in vesicles when monocytic cells enter M phase and free hypoacetylated HMGB1 into the cytoplasm after nuclear membrane breakdown.

FIG. 7. ERK Phosphorylation Directly Activates HMGB1 Acetylation and Vesicular Accumulation

U937.12 cells, when challenged with LPS for 3 hours, deplete their nuclear stores of HMGB1 and accumulate it in vesicles. Addition of 150 nM leptomycin together with LPS blocks HMGB1 relocation almost completely, and addition of 1 μM U0126 (sufficient to inhibit ERK1/2 phosphorylation and nuclear transfer, not shown) blocks it completely (compare to resting monocytes not challenged with LPS). In contrast, addition of 10 μM SB203580 (sufficient to inhibit p38 kinases completely, not shown) has no effect. Inhibition of protein synthesis in U937.12 with 10 μM cycloheximide (starting 20 minutes prior to LPS addition) does not block HMGB1 translocation, indicating that this process does not require gene expression.

FIG. 8. The Control of HMGB1 Secretion in Inflammatory Cells

In all cells, including resting inflammatory cells (left panel), HMGB1 partitions between nucleus and cytoplasm: nuclear import is mediated by the NLSs, and the protein re-diffuses back to the cytoplasm through the nuclear pores via passive diffusion and CRM1-mediated active export. When the NLSs are not acetylated, the rate of nuclear import exceeds that of rediffusion or export, and the protein appears predominantly or solely nuclear. Upon stimulation of inflammatory cells (right panel) through binding of IL-1β, TNF-α, LPS or HMGB1 itself to their own receptors, the NF-κB (not shown) and MAP kinase pathways are activated. The involvement of ERK1/2, but not p38 or Jnk, is demonstrated by the inhibition of HMGB1 translocation by U0126, but not SB203580 or SP600125. Phosphorylated ERKs migrate to the nucleus, where directly or via adaptor proteins they activate histone acetylases, or inhibit histone deacetylases. This in turn promotes the acetylation of the 2 NLSs of HMGB1. Exported acetyl-HMGB1 cannot return to the nucleus. Myeloid cells are equipped with a special variety of lysosomes that can be secreted upon appropriate stimulation (Andrews, 2000), and that can accumulate IL-1β (not shown) or HMGB1, presumably through the action of specific transporters embedded in the lysosomal membrane (Andrei et al., 1999; Gardella et al., 2002). Upon the binding of LPC (lysophosphatidylcholine, an inflammatory lipid) to its own receptor, the secretory lysosomes carrying HMGB1 fuse with the plasma membrane and secrete their cargo (Gardella et al., 2002).

### EXAMPLES

HMGB1 is unique among nuclear proteins in that it can be secreted, both by cells of myeloid origin (which use it as a proinflammatory cytokine) and by developing neural cells (where its role is much less understood) (reviewed by Müller et al., 2001b, and Bustin, 2002). The present work establishes that the alternative subcellular locations of HMGB1, in the nucleus where it serves its primary function as an architectural factor, and in the cytoplasm or in cytoplasmic vesicles, which serve as an intermediate station towards its eventual secretion, depends from the acetylation status of its nuclear localization sequences (NLSs). In U937 promyelocytic cells, the activity of HMGB1 acetyltransferases (or deacetylases), in turn, is regulated directly by ERK kinases.

### Example 1

### Protein Expression and Purification

Expression and purification of bacterially produced full-length HMGB1 and fragments thereof were performed as described (Müller et al., 2001a). HMGB1 was purified from calf thymus according to the protocol kindly provided by J. Bernués (CSIC, Barcelona). Briefly, the organ is minced in Buffer 1 (0.14 M NaCl, 0.5 mM EDTA, 0.1 mM PMSF), after removing the fat and the connective tissue. The homogenate is subjected to three 5% PCA extractions; supernatants collected after centrifuging are pooled and clarified with TCA (18% final concentration). Fractionated precipitations with acetone-HCl (400:1 v/v) and acetone alone eliminate histone H1. The sediment, dissolved in borate buffer pH 9.0 is then passed once or twice (according to the purity of the sample) on a CM-Sephadex C25 column, and eluted with a NaCl gradient from 0.11 M to 0.2 M.

Throughout this specification, residues in HMGB1 have been numbered according to Allfrey and coworkers: first aa of mature HMGB1 is a glycine, since the first methionine encoded by the gene is cleaved off after synthesis.

### Eample 2

### Plasmids

Plasmid pEGFP-HMGB1 contains the open reading frame of HMGB1 fused at the 3′ end with the coding region of the Enhanced Green Fluorescent Protein (EGFP), as described previously (Scaffidi et al., 2002). Plasmid pEGFP-HMGB1 was used as template to generate the mutants in NLS1 and NLS2 in two-step PCR mutagenesis, using 5′HMG-GFP (5′-ATCCTCgAgACATgggCAAAggAg-3′) and 3′HMG-GFP (5′-ACCCCgCggTTCATCATCATCATC-3′) as external primers and six pairs of internal mutagenic primers (substitutions in bold type):

NLS1KQdir 5′-gAggAgCACCAgCAgCAgCACCCggATg-3′
NLS1KQrev 5′-CATCCgggTgCTgCTgCTggTggTCCTC-3′;
NLS1KRdir 5′-CACAggAggAggCACCCggATgCTTCTgTC-3′
NLS1KRrev 5′-gTgCCTCCTCCTgtgCTCCTCCCgGCAg-3′;
NLS1KAdir 5′-gAggAgCACgCggCggCgCACCCggATgC-3′
NLS1KArev 5′-gCATCCgggTgCgCCgCCgCgTgCTCCTC-3′;
NLS2KQdir 5′-AgCCAgCAACAgAAggAAgAggAAgACgACgAg-3′
NLS2KQrev 5′-CTTCTgTTgCTggCTCTTCTCAgCCTTgAC-3′;
NLS2KRdir 5′-AgCAggAgAAggAAggAAgAggAAgACgACgAg-3′
NLS2KRrev 5′-CTTCCTTCTCCTgCTCTTCTCAgCCTTgAC-3′;
NLS2KAdir 5′-AgCgCggCAgCgAAggAAgAggAAgACgAC-3′
NLS2KArev 5′-CgCTgCCgCgCTCTTCTCAgCCTTgAC-3′.

The final PCR products were then cloned into pEGFP-HMGB1 cut with XhoI and SacII to obtain the mutant plasmids.

Double mutants were generated using the NLS2 internal mutating primers and the NLS1 mutants of pEGFP-HMG1 as template. The cloning approach was the same as for the single mutations.

We generated the constructs for NLS-GFP and NLS2-GFP fusions by cloning between the XhoI/SacII sites of the pEGFP-N1 vector two cassettes, produced by annealing the following pairs of oligonucleotides:

NLS1dir:
5′-TCTACTCgAgACATGAAgAAgAAgCACCCggATgCTTCTgTCAACTT
CTCAgAgTTCTCCAAgAAgCCgCggCTAA-3′
and
NLS1rev:
5′-TTAgCCgCggCTTCTTggAgAACTCTgAgAAgTTgACAgAAgCATCC
gggTgCTTCTTCTTCATgTCTCgAgTAgA-3′;
NLS2dir:
5′-TCTACTCgAgACATGAAgAgCAAgAAAAAgAAggAACCgCggCTCA-
3′
and
NLS2rev:
5′-TgAgCCgCggTTCCTTCTTTTTCTTgCTCTTCTAgTCTCgAgTAgA-
3′.

All constructs were verified by sequencing.

### Example 3

### 2D Gel Electrophoresis

About 50 μg of purified HMGB1 or about 250 μg of total cellular protein were added to 350 μl of rehydration buffer (RB), containing 8 M urea, 2% CHAPS, 20 mM dithioerythritol (DTE), 0.8% IPG buffer (carrier ampholytes, pH 3-10 non-linear or pH 4-7 linear). Samples were applied on 18 cm polyacrylamide gel strips (pH range: 3-10 NL, or pH 4-7 L). Isoelectrophocusing (IEF) was performed in IPGphor (Pharmacia Biotech). IEF was stopped at 75000-90000 VoltHours. Second dimension runs were performed using a Protean II apparatus (Bio-Rad). After IEF, strips were soaked first in equilibration buffer (EB: 6 M urea, 3% SDS, 375 mM Tris pH 8.6, 30% glycerol, 2% DTE), then in EB containing 3% iodoacetamide (LAA) and traces of bromophenol blue (BBP). Strips were then applied onto 10%-12% polyacrylamide gels. Gels were run at 90 V for about 16 hours, and were then either silver stained or transferred onto nitrocellulose membranes (ECL, Amersham) in 25 mM Tris pH 7.5, 0.192 M glycine, 20% methanol.

### Example 4

### Mass Spectrometry

Isolated protein spots were excised from 2D gels stained with colloidal Coomassie, and reduced and alkylated as described (Shevchenko et al., 1996). We then performed sequential digestions using different sequence specific proteases and/or chemical cleavage. In particular, Trypsin, Asp-N, Glu-C digestions were performed in 50 mM NH₄HCO₃ buffer pH 8.0 at 37° C., with shaking. Time of digestion varied from 3 hours to overnight according to the efficiency of cleavage. Chemical cleavage with formic acid (Asp-C) was achieved by incubating spots overnight at 56° C. in 2% formic acid. Spots blotted on PVDF membranes were incubated in the presence of cyanogen bromide (CNBr) in 70% trifluoroacetic acid for 1 hour at RT in the dark. One μl of digestion products was loaded onto the MALDI target using the dried droplet technique and α-cyano-4-hydroxycinnamic acid (HCCA) or sinapinic acid as matrix.

MALDI-TOF mass measurements were performed on a Voyager-DE STR time of flight (TOF) mass spectrometer (Applied Biosystems, Framingham, Mass., USA) operated in the delayed extraction and reflector mode. Spectra, internally calibrated, were processed via the Data Explorer software.

### Example 5

### Cell Cultures, Transfections, and Treatments with LPS and Inhibitors

Fibroblasts and HeLa cells were cultured in Dulbecco's modified Eagle's medium (DMEM) supplemented with 10% fetal bovine serum (FBS, from Gibco), 100 IU/ml of penicillin and 100 μg/ml streptomycin, in 5% CO₂ humidified atmosphere. HeLa cells and 3134 mouse fibroblasts were transfected by calcium phosphate co-precipitation. To express fluorescent proteins, 3×10⁵ cells were plated in 6 cm dishes and were transiently transfected with 8 μg of the appropriate plasmid. Cells were observed 36 hours after transfection. The average amount of HMGB1-GFP in the cell population was between 1 and 3% of HMGB1 (as measured by immunoblotting with anti-HMGB1 antibodies). About 24 hours after transfection, 3134 fibroblasts were treated for 3 hours with 10 ng/ml trichostatin A (TSA, Sigma) before fixing and imaging. Subclone 12[−] of U937 promoyelocitic cells, kindly provided by M. Alfano and G. Poli (HSR, Milano), was grown in RPMI medium (Gibco) supplemented with 10% FCS, L-glutamine, 100 U/ml penicillin 100 μg/ml streptomycin, exposed for 3 days to 0.1 μM vitamin D3 (Roche) to promote CD14 expression, and stimulated with 100 ng/ml LPS (Sigma catalogue #L4391) or with 10 ng/ml TSA for 3 hours. For the experiments with kinase inhibitors, cells were exposed to 1 μM U0126, 10 μM SB203580 or 30 μM SP600125 (all from Calbiochem) at the same time as LPS, and lasting for 3 hours. Primary human monocytes, purified from peripheral blood (a kind gift from M. Iannacone, HSR), were maintained in RPMI medium supplemented as described above, and were activated with 100 ng/ml LPS.

### Example 6

### Heterokaryon Assays

HeLa cells were plated on glass coverslips in a 6-well dish (100 000 cells/dish). After 16 hours, 200 000 Hmgb1−/− mouse fibroblasts were plated on the same coverslips. Following a 3-hour incubation in the presence of 100 μg/ml cycloheximide, the cells were washed with PBS and treated with 100 μl of prewarmed 50% PEG-6000 in PBS for 1 min. After 3 washes with PBS, the cells were incubated with DMEM containing 100 μg/ml cycloheximide for 4 hours, and then fixed with 4% paraformaldehyde. Immunofluorescence was performed using anti-human cytokeratin (Santa Cruz) and anti-HMGB1 antibodies, and chromatin was visualized by DAPI staining. When indicated, 150 nM leptomycin B (a kind gift of Barbara Wolff, Novartis, Vienna) was added to the medium together with cycloheximide.

For testing passive diffusion of HMGB1, HeLa cells were pretreated with 100 μg/ml cycloheximide at 37° C. for 30 min, incubated at 4° C. for 4 hours, fixed with 4% paraformaldehyde, and stained with anti-HMGB1 antibodies.

### Example 7

### Pull Down Assays with CRM1

CRM1 protein was in vitro transcribed-translated with the TnT Coupled Reticulocyte Lysate System (Promega) following the manifacturer's protocol, using pSGCRM1 plasmid as template. Seven μl of freshly made [³⁵S]-Met labeled CRM1 were incubated in 15 μl RAN Buffer (50 mM Tris-HCl pH 7.5, 200 mM NaCl, 2 mM MgCl₂, 10% glycerol), 5 μl of 6×CRM1 buffer (20 mM HEPES-KOH pH 7.5, 80 mM CH₃COOK, 4 mM Mg (CH₃COO)₂, 250 mM sucrose, 2.5 mM DTT), 1 mg/ml BSA, 400 nM Leptomycin B where indicated, and approximately 10 μl of beads bearing immobilized GST-NS2, BSA, recombinant tailless HMGB1 (HMGB1ΔC, Muller et al., 2001b), boxA, or boxB. GST-NS2 was coupled to Gluthatione Sepharose (Amersham), the other proteins were covalently cross-linked to Activated Sepharose-CH (Amersham). The incubation was for one hour at 4° C. on a rotating wheel. The beads were pelleted by centrifugation, supernatants were dried in Savant. Beads were then washed five times at 4° C. with 50 volumes of PBS containing 9% Glycerol, 5 mM MgCl₂ and 1% NP-40. Beads were then boiled in 10 μl SDS-PAGE loading buffer, and loaded on a 8% SDS PAGE together with dried supernatants (output), the fourth wash (W4) and equivalent amount of input as a reference. The gel was then blotted on nylon filter and exposed to X-ray film to detect labeled CRM1.

### Example 8

### Immunofluorescence and GFP Imaging

Cell cultured in LabTek II chambers (Nalgene) were directly fixed in 3.7% paraformaldehyde (PFA) in PHEM buffer (36.8 g/l PIPES, 13 g/l HEPES, 7.6 g/l EGTA, 1.99 g/l MgSO4, buffered to pH 7.0 with KOH) for 10 minutes at room temperature. After fixation, cells were washed with PBS and incubated for 3 minutes at 4° C. with HEPES-based permeabilization buffer containing 300 mM sucrose and 0.2% Triton X-100. Fifteen minutes of incubation in blocking solution (BlS, 0.2% BSA in PBS) followed. Primary antibodies were then diluted in BIS to the suitable final concentration and incubation was prolonged for 1 hour at room temperature. After 3 rinses with BlS, cells were incubated with secondary antibodies in BIS for one hour, washed three times with BlS, and then incubated with PBS containing 0.5 μg/ml Hoechst 33342. The polyclonal rabbit anti-HMGB1 was purchased from BD PharMingen (Torrey Pines, Calif.), and used at 1:1600 dilution. Goat polyclonal antibodies against rabbit IgG (H+L) conjugated to Alexa Fluor 594 (working dilution 1:1000) were purchased from Molecular Probes (Eugene, Oreg., USA).

Cells expressing HMGB1-GFP, its derivatives and the NLSs-GFP fusions were PFA-fixed as described above, then incubated in PBS containing Hoechst 33342 to stain the nuclei and finally imaged.

Cells were imaged using Olympus 60× or 100× 1.4NA Plan Apo oil immersion objective lenses on a DeltaVision Restoration Microscopy System (Applied Precision, Issaquah, Wash., USA) built around an Olympus IX70 microscope equipped with mercury-arc illumination. Filters were from Chroma Technology Corp. (Brattleboro, Vt., USA): Hoechst 33342 excitation 360/40, emission 457/50; GFP excitation 490/20, emission 528/38; AlexaFluor 574, excitation 555/30, emission 617/73. Forty optical sections spaced by 0.4 μm were collected with a Coolsnap_Hq/ICX285 CCD camera (Photometrix, Tucson, Ariz., USA) and deconvolved by the constrained iterative algorithm available in the SoftWoRx 2.50 package (Applied Precision) using 10 iterations and standard parameters. Each image measured 512×512 pixels, and effective pixel size was 106 nm (60×) or 63 nm (100×).

### Example 9

### Bioinformatics

HMGB1 was inspected for potential NLSs using the PredictNLS database, maintained at CUBIC (http://cubic.bioc.columbia.edu/predictNLS) (Cokol et al., 2001). PredictNLS is an automated tool for the analysis and determination of Nuclear Localization Signals (NLS). By submitting a protein sequence or a potential NLS, PredictNLS predicts if the protein is nuclear, or finds out whether the potential NLS corresponds to a known one in the database. The program also compiles statistics on the number of nuclear/non-nuclear proteins in which the potential NLS is found or has a match. Finally, proteins with similar NLS motifs are reported, and the references to the experimental papers describing the particular NLS are given.

### Example 10

### 2D Electrophoresis Resolves Several Spots Corresponding to HMGB1 Protein

HMGB1 was already known to be acetylated on lysines 2 and 11 (Sterner et al., 1979), and to be ADP-ribosylated. We re-examined the issue of post-translational modifications of HMGB1 using bi-dimensional electrophoresis. To expand the variety of modifications surveyed, we analyzed HMGB1 from thymus, which is a complex organ comprising cells of the lymphoid, myeloid and epithelial lineages.

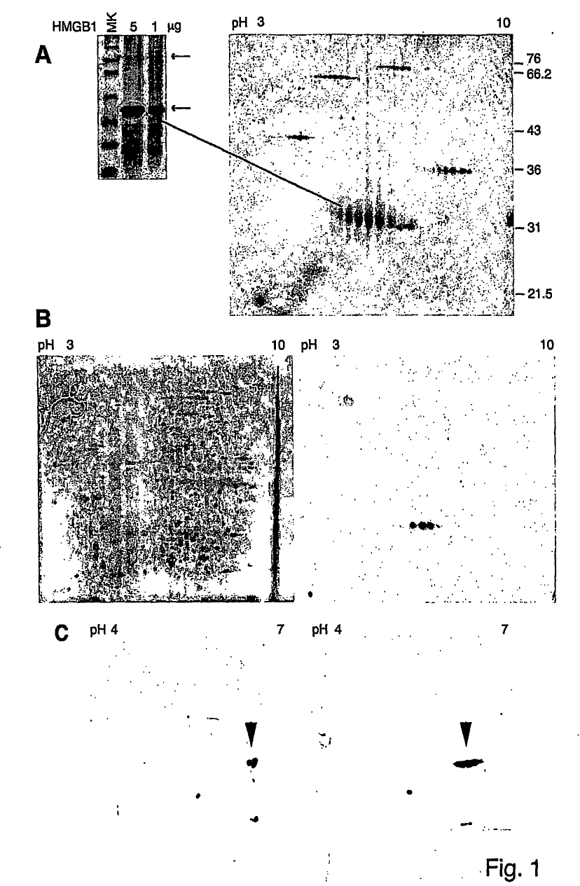

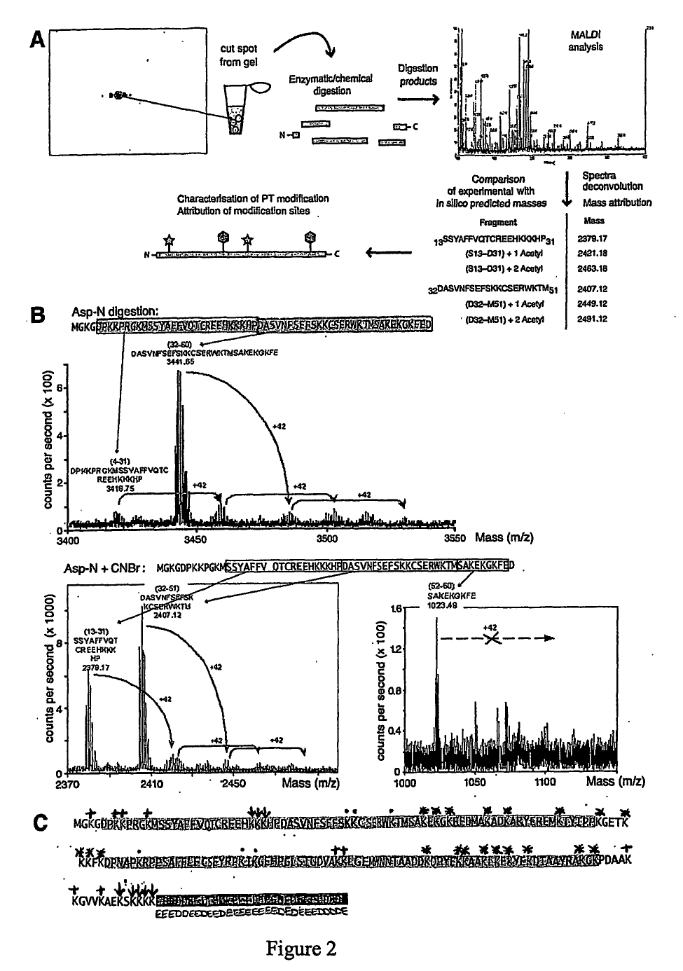

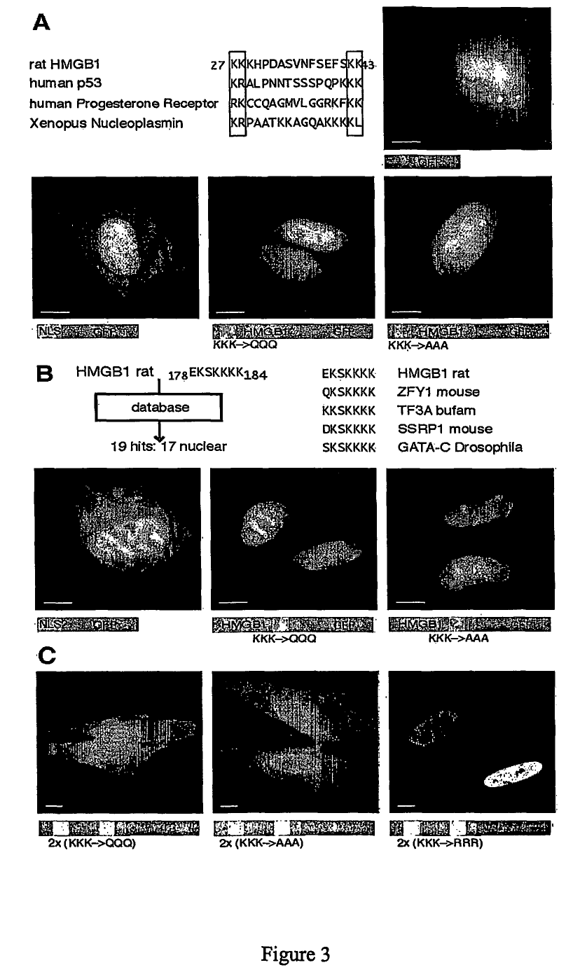

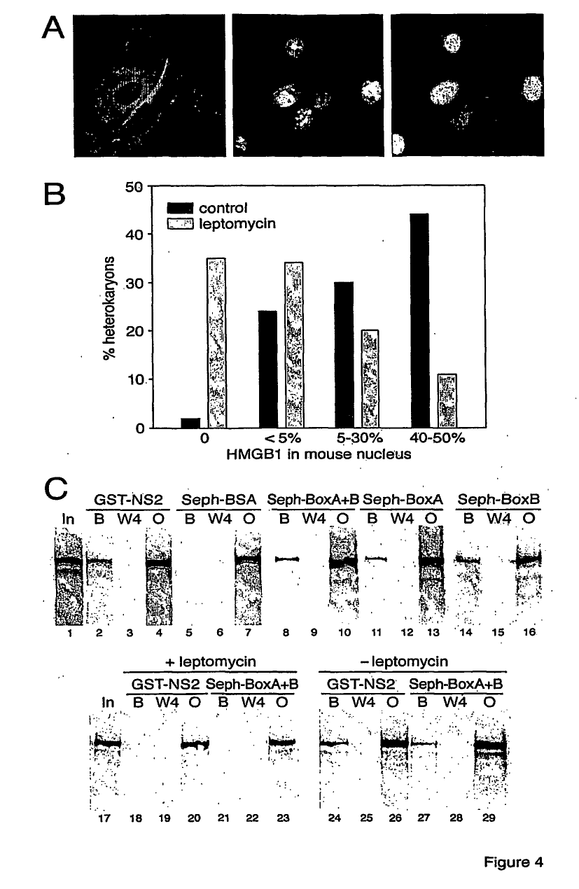

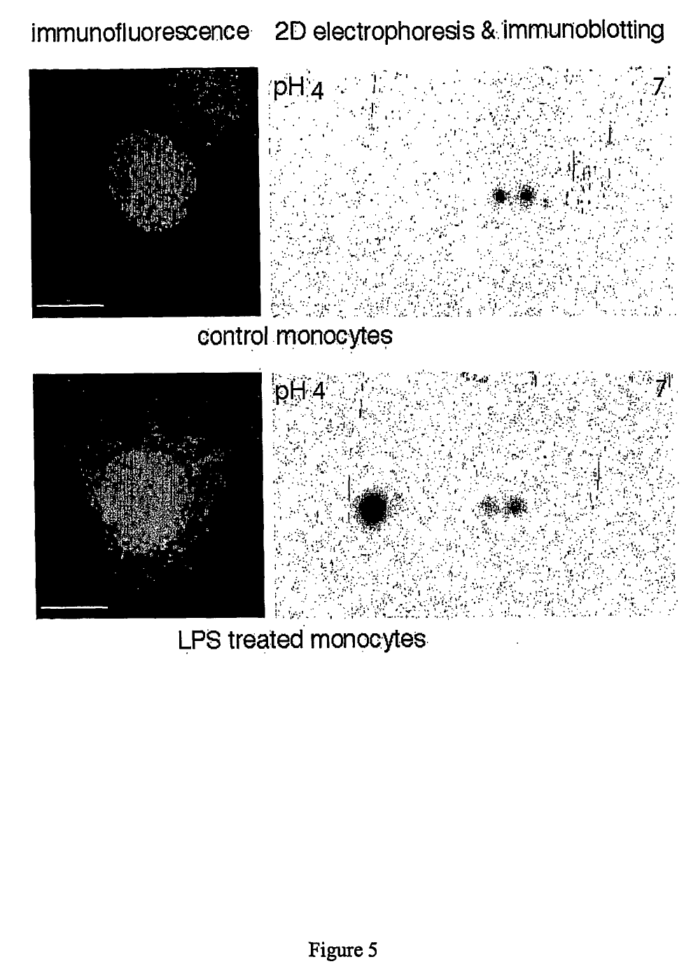

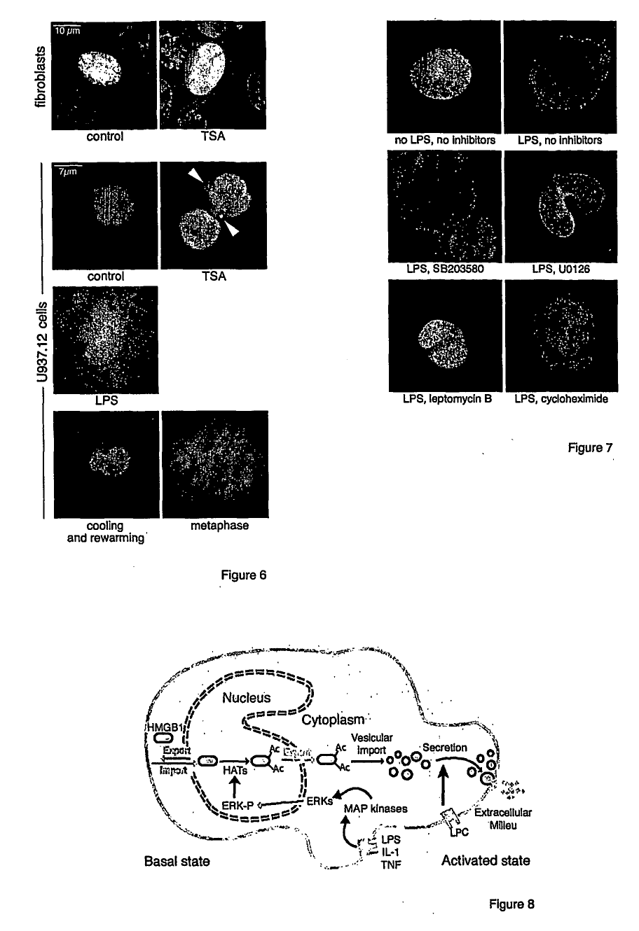

Purified HMGB1 separated in 2 bands in mono-dimensional SDS-PAGE, a major one corresponding to an apparent molecular weight of 29 kDa, and a minor one containing HMGB1 modified with ADP-ribosyl moieties (FIG. 1A, left gel, and results not shown). When 50 μg of this preparation were separated by 2D electrophoresis and silver-stained, 8 to 10 different spots appeared (FIG. 1A, right). The spots were regularly spaced along the pH gradient, and had very similar molecular weight, suggesting that the modifications were affecting the charge of the molecule. A polyclonal anti-HMGB1 antibody recognized all the spots with similar affinity after blotting onto nitrocellulose filters (data not shown); this confirmed that they all corresponded to different isoforms of the same protein.

To exclude the possibility that the spots might arise from purification artefacts, we prepared a total extract of thymus from a 17-day mouse embryo and analyzed it in the same way. Twin gels were loaded; one was silver stained and the other blotted and probed with anti-HMGB1 antibodies: multiple spots for HMGB1 appeared, confirming that the modifications are physiological (FIG. 1B). The smaller number of spots detected was probably due to the lower quantity of HMGB1 loaded, given the complexity of the whole-tissue extract.

We also verified the acetylation status of HMGB1 in 3 different cell lines (3T3 mouse fibroblasts, HeLa cells, HEK cells). These showed only two isoforms of HMGB1 (FIG. 1C, left), consistent with previous findings. However, even in cell lines with a low complexity of HMGB1 acetylation, hyperacetylation could be obtained by treatment with general deacetylase inhibitors, like trichostatin A (TSA), sodium butyrate, and HC-toxin (FIG. 1C right, and results not shown). HDAC inhibitors generated a train of spots shifted to the more acidic part of the pH gradient, similar to the pattern detected with HMGB1 purified from calf thymus. The maximal level of acetylation was reached between 5 and 6 hours after treatment (data not shown).

### Example 11

### HMGB1 is Multiply Acetylated

Mass spectrometry (MS) was used to investigate the nature of the modifications occurring in HMGB1. MS analysis determines the exact mass of protein fragments, allowing the identification of proteins according to their specific fragmentation profiles; alternatively, it can characterize protein modifications by estimating accurately the difference between the experimentally found and the predicted masses of peptides.

We excised the four most abundant spots from Coomassie-stained 2D gels and digested the protein with trypsin, a protease that cleaves peptide bonds at the C-terminus of lysines and—to a lower extent—of arginines. Peptide masses were determined by MALDI-TOF, and the mass corresponding to each digestion product was compared with the mass expected from the in silico digestion (FIG. 2A).

We found that the mass difference among peptides was frequently 42 Da, or a multiple of this value, that corresponds to acetyl moieties. Proteins are acetylated by the condensation of an acetyl group (—COCH₃) to the ε amino group of lysines; this causes the neutralization of one basic charge and the decrease of the pI of the entire protein. The 2D pattern of spots with even spacing and little molecular weight change is readily explained by multiple acetylations, and the presence of up to 10 spots suggests that at least as many lysines can be acetylated.

Even today, the analysis of protein mixtures with 10 or more acetylation sites is challenging. Furthermore, HMGB1 contains 43 lysines over a length of just 214 amino acids, and many are clustered within a single proteolytic fragment.

We devised a multiple-digestion strategy: we used different site-specific proteolytic agents (Asp-N, CNBr, trypsin, Glu-C, Asp-C), both singly and in combination, and analyzed the peptides by MALDI. Thus, one or more acetylations were assigned to large fragments of the protein (but not to specific lysines), and their positions were then progressively restricted inside smaller fragments. In the example provided in FIG. 2, HMGB1 was first digested with Asp-N, and the non-acetylated fragments expected from the digestion were identified in the complex pattern of MS data. Peaks corresponding to the masses of the non-acetylated peptides plus 42 or n-multiples of 42 were presumptively assigned as 1-to-n acetylations of that specific peptide. Then, aliquots of the same peptides were further cleaved with CNBr, and smaller fragments were obtained. The assignment procedure was iterated, until the acetylations could be attributed to specific lysines.

By analysis of a large set of digestion products (not shown), we were able to assign 17 acetylated lysines and to exclude modification of 20 lysines; only 6 lysines out of 43 remained uncharacterized. We could not find evidence of other types of modification; in particular, we sought for methylation, phosphorylation and glycosylation. MS is not a quantitative technique, but it was very clear from the data that no lysine was acetylated in all HMGB1 molecules in the sample. In principle, if each of the lysines we assigned can be acetylated independently, there can exist 2¹⁷ (over 100 000) molecular species of HMGB1; however, it appeared that several lysines tend to be acetylated as a cluster.

### Example 12

### HMGB1 has a Bipartite Nuclear Localization Signal

Examination of the acetylated clusters suggested that the lysines within residues 27 and 43 of HMGB1 might represent a Nuclear Localization Signal (NLS). This sequence is a perfect match to the characteristic consensus of classical bipartite NLSs (Cokol et al., 2001), usually composed of two stretches of 2-3 basic residues (K or R), separated by about 12 amino acids (FIG. 3A).

We fused aa 27-43 of HMGB1 to the N-terminus of GFP, and compared it to transiently expressed GFP in HeLa cells. Unfused GFP was distributed all over the cell, whereas about 90% of fluorescence from the NLS-GFP fusion was concentrated in the nucleus (FIG. 3A). Thus, amino acids 27-43 can provide a functional NLS for HMGB1.

Since lysines 27, 28, 29 are among the most frequently acetylated, we examined the possibility that acetylation of the NLS was related to the sub-cellular localization HMGB1. We mutated lysines 27-29 of the HMGB1-GFP fusion into glutamines to mimic acetylation; we also fully destroyed the NLS by changing the 3 lysines into alanines. Surprisingly, neither mutation affected the nuclear localization of HMGB1-GFP, suggesting that an element other then aa 27-43 must contribute to locate HMGB1 into the nucleus (FIG. 3A).

### Example 13

### A Second NLS Can Drive HMGB1 to the Nucleus

By visual inspection of the HMGB1 sequence we could not find any other canonical NLS, so we took advantage of the database available at http://cubic.bioc.columbia.edu/predictNLS, which offers a collection of experimentally verified plus in silico-generated NLSs (Cokol et al., 2001), to help us predict additional NLSs in HMGB1.

The region between aa 178-184 appeared as a good candidate for a monopartite NLS motif: typically, these are characterized by a cluster of basic residues preceded by a helix-breaking residue, but some variations are possible (Boulikas, 1993). When this sequence was launched in the database, it was not identified as an already known NLS, but it matched at highest similarity with several proteins, 93% of which were nuclear (FIG. 3B). We tested its NLS activity by fusing it to the N-terminus of GFP: the fusion protein showed about 90% of the fluorescence in the nucleus (FIG. 3B). Thus, HMGB1 is endowed with 2 motifs with NLS activity: we called the bipartite motif NLS 1 and the monopartite motif NLS2. Remarkably, NLS2 too corresponds to a cluster of frequently acetylated lysines.

When lysines 181, 182, 183 of the HMGB1-GFP fusion were changed into glutamines or alanines, the result was similar to that obtained with NLS1 mutations: NLS2 mutants showed no re-localization into the cytoplasm (FIG. 3B). Double mutants of HMGB1-GFP, where both lysine clusters in NLS 1 and NLS2 are changed to glutamines, alanines or arginines, were then constructed. The double NLS1/NLS2 mutant where lysines are changed to arginines remains nuclear. Double K->Q and K->A mutants have clearly a cytoplasmic localization (FIG. 3C); the fusion proteins are also present within the nucleus, suggesting that they are small enough to pass through the nuclear pores by passive diffusion.

### Example 14

### HMGB1 has Non-classical Nuclear Export Signals

To test whether HMGB1 (mw 25 kDa) can pass to and from the nucleus by simple diffusion across the nuclear pores, we incubated HeLa cells at 4° C. for 4 hours, a condition that impedes active, GTP-driven nuclear import/export. After cold incubation, of a part of HMGB1 diffused back to the cytoplasm; the same was true for HMGB1-GFP (not shown).

However, many small proteins that are able to cross the nuclear membranes by passive diffusion are also endowed with a Nuclear Export Signal (NES), that promotes active nuclear extrusion of the protein. To test for active export, Hmgb1−/− mouse embryonic fibroblasts (Calogero et al., 1999) were fused with HMGB1-positive human HeLa cells. Human cytoplasm was identified by staining with anti-human cytokeratin antibodies (red, FIG. 4A), mouse nuclei by their bright DAPI-positive heterochromatic whitish spots over a blue background. Cells with human cytokeratin and a mouse nucleus were heterokaryons. HMGB1 is 99.5% identical in mouse and human, and its nuclear presence was scored with green fluorescent antibodies. Shortly after fusion, mouse nuclei in heterokaryons had very little HMGB1 (not shown), but 4 hours at 37° C. (in the presence of cycloheximide to inhibit protein synthesis) were generally sufficient to equilibrate evenly HMGB1 between the human and the mouse nucleus (FIGS. 4A and B). By itself, this result just indicates that HMGB1 can traverse the nuclear membranes from the nuclear side to the cytoplasmic side, and does not show that the export is active. However, active export that involves the CRM1 exportin can be inhibited by leptomycin B. We then treated heterokaryons with leptomycin, and noticed that re-equilibration of HMGB1 between human and mouse nuclei was strongly reduced (FIG. 4B). Quantitation of the intensity of green fluorescence showed that in the vast majority of leptomycin-treated heterokaryons less than 5% of HMGB1 had migrated to the mouse nucleus after 4 hours (in the controls, almost 50% of nuclei had equilibrated).

These results indicate that HMGB1 has a NES that interacts with the CRM1 exportin (the target of leptomycin). We then synthesized labeled CRM1 in a reticulocyte extract, and passed it over columns that contained NS2 (a strong nuclear signal as positive control), BSA (a negative control), tailless HMGB1 (BoxA+B), BoxA or BoxB. CRM1 bound to both HMG boxes, indicating that each contains an NES. When 400 nM leptomycin was added to the reticulocyte extract containing CRM1, the labeled protein did not bind to the either NS2 or BoxA+B, that contains both NESs.

### Example 15

### HMGB1 Secreted by LPS-Activated Monocytes is Hyperacetylated

Resting monocytes obtained from human peripheral blood contain nuclear HMGB1; upon challenge with LPS, monocytes accumulate a major portion of HMGB1 into cytoplasmic vesicles (FIG. 5), while the nuclear protein is progressively depleted (not shown). Translocation takes about 16 hours (with variability associated to different donors), and is not inhibited by cycloheximide (not shown). HMGB1 has no leader peptide; moreover, HMGB1 secretion does not follow the classical ER-Golgi pathway (Gardella et al., 2002). The coincidence between NLSs and acetylation clusters in the HMGB1 purified from thymus suggested that vesicular accumulation might follow from cytoplasmic localization, and this would depend from the acetylation of NLSs.

We generated total extracts from resting and activated monocytes and analyzed them by 2D electrophoresis and western blotting. Remarkably, while resting monocytes show only two isoforms of HMGB1, like all cell lines we tested so far, in LPS-activated monocytes the redistribution of HMGB1 into cytoplasmic vesicles parallels the appearance of a major additional HMGB1 spot (FIG. 5). The spot is approximately at the same molecular weight of the two “baseline” ones, but at far lower pI. The software ImageMaster 2D, specifically designed to calculate the pI of a spot from its position in a gel and to estimate the number of specific modifications corresponding to a defined ΔpI, predicts that the novel spot corresponds to HMGB1 acetylated 4 or 5 times more than the rightmost baseline spot. The absence of intermediate spots between the “activated” and baseline spots suggests that in monocytes the acetylation of HMGB1 is not gradual, but highly concomitant.

### Example 16

### Acetylation of HMGB1 Determines its Relocation to the Cytoplasm in Fibroblasts and to Vesicles in Promyelocytic Cells

The results showed above provided a correlation between acetylation of HMGB1 and its sub-cellular localization. To further confirm the role of acetylation, we performed experiments with fibroblasts, that do not contain secretory lysosomes, and with U937 promyelocytic cells, that do contain them.

TSA treatment for 3 hours of 3134 mouse fibroblasts transiently expressing GFP-HMGB1 was sufficient to relocate a significant amount of HMGB1 to the cytoplasm (FIG. 6), suggesting that hyperacetylation can force HMGB1 to relocate to the cytoplasm in most cells.

Different subclones of U937 cells perform differently in cytokine secretion; for this reason, we chose a defined subclone, 12[−], with a well-established profile of plasma membrane molecular markers and functional responses (Biswas et al., 2001; Bovolenta et al., 1999). U937.12 cells paralleled closely the behavior of primary monocytes: resting cells contained HMGB1 in the nucleus, whereas LPS-activated ones redistributed a significant fraction of the protein into cytoplasmic vesicles (FIG. 6). However, the relocation was much faster than in monocytes, and was clearly visible starting 1 hour after stimulation.

To test whether HMGB1 localization was directly determined by its acetylation status, we treated U937.12 cells with 10 ng/ml TSA for 3 hours: a fraction of HMGB1 started to shift to cytoplasmic vesicles (FIG. 6, arrows). Remarkably, this occurred in the absence of the cytoplasmic expansion that accompanies activation by LPS.

We also tested whether hypoacetylated HMGB1 could be taken up by secretory lysosomes: we incubated U937.12 cells for several hours at 4° C., causing the passive diffusion of a significant fraction of HMGB1 to the cytoplasm, and then raised back the temperature to 37° C. Within 5 minutes, HMGB1 was accumulated in secretory lysosomes. Likewise, HMGB1 liberated into the cytoplasm by the breakdown of the nuclear membrane during mitosis (Falciola et al., 1997) was also accumulated into secretory lysosomes.

HMGB1-GFP, expressed in stably transfected U937.12 clones, was shuttled from nucleus to cytoplasm in response to LPS and TSA, but was unable to proceed further into secretory lysosomes, and much less to be secreted (not shown). Double NLS1/NLS2 mutants (double K->A and double K->Q) of HMGB1-GFP were present in the cytoplasm resting U937 cells, confirming their phenotype in fibroblast cells (not shown). The double substitution from lysines to arginines was lethal to U938.12 cells, and could not be tested.

### Example 17

### Acetylation of HMGB1 is Controlled via the ERK MAP Kinase Pathway

The experiments reported above indicate that U937.12 cells acetylate and accumulate HMGB1 in vesicles in response to activation. Therefore, acetylation or deacetylation activities (at least of HMGB1) must be controlled by signal transduction pathways that start with the engagement of receptors for TNF-α, IL-1β, LPS, HMGB1 itself and/or other pro-inflammatory ligands. The secretion of TNF-α, IL-1, IL-8, and PGE₂ by LPS-stimulated monocytes can be blocked by inhibiting ERK phosphorylation (Scherle et al., 1998). We therefore tested whether the same was true for HMGB1 acetylation and vesicular accumulation. U937.12 cells were exposed to 200 ng/ml LPS in the presence of 1 μM U0126 (a specific inhibitor of ERK phosphorylation), 30 μM SP600125 (a specific inhibitor of Jnk kinases) or 10 μM SB203580 (a specific inhibitor of the p38 kinases). Control cultures were not exposed to LPS, or exposed to LPS but no inhibitors. HMGB1 translocation was substantial in cells exposed to LPS alone, or LPS plus inhibitors of Jnk (not shown) and p38, was partially blocked in cells exposed to leptomycin B, and was almost completely blocked in cells exposed to LPS plus U0126 (FIG. 7; more than 95% of the cells had the morphology shown). All kinase inhibitors had no effect on U937.12 cell viability, nor on adhesion and cytoplasm expansion that follow activation; HMGB1 translocation after LPS stimulation was restored after the removal of U0126 (not shown).

These results indicate that ERK kinases (but not p38 or Jnk kinases) are involved in the control of HMGB1 acetylation and translocation. ERK1 and 2 migrate to the nucleus when phosphorylated by the kinase MEK1 (the step that is inhibited by U0126), and in turn phosphorylate several transcription factors that promote the expression of a number of genes. For example, secretion of TNF-α involves transcriptional upregulation of the TNFA gene, which depends from the expression of the EGR1 transcription factor, which in turn depends form the ERK-mediated phosphorylation of the transcription factor ELK1 (Guha et al., 2002). In contrast, HMGB1 relocation does not depend from LPS-activated expression of specific genes: U937.12 cells kept in the presence of 10 μg/ml cycloheximide still translocate HMGB1 to vesicles when LPS is added (FIG. 7). The same was true for human monocytes (although on a time scale of 16-20 hours). These results indicate that the activation of HMGB1 acetylation only requires post-translational modification of pre-existing proteins.

### Example 18

### Purification of Polyclonal Antibodies Specific for the Acetylated Form of HMGB1

Two oligopeptides (Pep1, PKGETKKKFKD, comprising the acetylatable lysines in NLS1, and Pep2, AKKGVVKAEKSKKKKE, comprising NLS2) were synthesized by Tecnogen (Piana di Monte Vema, Italy), together with their companions AcPep1 and AcPep2, in which all lysines were substituted by acetyl-lysines. The identity, composition and purity of the four peptides were checked by RP-HPLC and MALDI-TOF. The peptides were then covalently linked to Activated CH Sepharose 4B (Amersham Pharmacia Biotech), following the manufacturer's instructions.

Rabbit polyclonal antibody raised against HMGB1 purified from calf thymus was passed first on the column with immobilized AcPep2. The bound antibody was eluted with 0.1 M glycine-HCl pH 2.0 and the eluate was immediately titrated to pH 7.0 with Tris base; BSA was added to 100 μg/ml. This solution was passed through the column with immobilized Pep2; the flowthrough was collected. This contained antibodies that recognized the acetylated peptide but not the non-acetylated peptide, and we called this preparation anti-AcNLS2. Anti-AcNLS1 was prepared in similarly.

Anti-NLS2 was used in an ELISA assay against recombinant HMGB1 purified from bacteria (non-acetylated) and HMGB1 purified from calf thymus (a mixture of different acetylated forms of HMGB1). The two forms of HMGB1 were recognized by anti-AcNLS2 with different affinity: the signal obtained from bacterially-made HMGB1 was at least 50 times larger than from calf thymus HMGB1. The same ELISA assay performed using the Pharmingen anti-HMGB1 antibody gave comparable signals for bacterially made and calf thymus HMGB1. Anti-NLS 1 was less specific, and recognized calf thymus HMGB1 only 4-5 fold better than bacterially-made HMGB1.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in molecular biology or related fields are intended to be within the scope of the following claims.

### REFERENCES—(HEREIN INCORPORATED BY REFERENCE)

1. Janeway, C. A., Immunol Today. 13, 11-16 (1992).
2. Banchereau, J. & Steinman, R. M., Nature 392,245-252 (1998).
3. Gallucci, S., Lolkema, M. & Matzinger, P., Nat Med. 5, 1249-1255 (1999).
4. Sauter, B., Albert, M. L., Francisco, L., Larsson, M., Somersan, S. & Bhardwaj, N. J., Exp. Med. 191, 423-434 (2000).
5. Ignatius, R., Marovich, M., Mehlhop, E., Villamide, L., Mahnke, K., Cox, W. I., Isdell, F., Frankel, S. S., Mascola, J. R., Steinman, R. M. & Pope, M. J., Virol. 74, 11329-11338 (2000).
6. Shi, Y., Ziheng, W. & Rock, K. L., Proc. Natl. Acad. Sci. U.S.A. 97, 14590-14595. (2000).
7. Basu, S., Binder, R. J., Suto, R., Anderson, K. M. & Srivastava, P. K., Int. Immunol. 12, 1539-1546 (2000).
8. Larsson, M., Fonteneau, J. F. & Bhardwaj, N., Trends Immunol. 3, 141-148 (2001).
9. Rovere, P., Vallinoto, C., Bondanza, A., Crosti, M. C., Rescigno, M., Ricciardi-Castagnoli, P., Rugarli, C. & Manfredi, A. A., J. Immunol. 161, 4467-4471 (1998).
10. Scaffidi, P., Misteli, T. & Bianchi, M. E., Submitted to Nature.
11. Yang, H., Wang. H. & Tracey K. J., Shock 15,247-253 (2001).
12. Andersson, U., Wang, H., Palmblad, K., Aveberger, A. C., Bloom, O., Erlandsson-Harris, H., Janson, A., Kokkola, R., Zhang, M., Yang, H. & Tracey, K. J., J. Exp. Med. 192, 565-570 (2000).
13. Wang, H., Bloom, O., Zhang, M., Vishnubhakat, J. M., Ombrellino, M., Che, J., Frazier, A., Yang, H., Ivanova, S., Borovikova, L., Manogue, K. R., Faist, E., Abraham, E., Andersson, J., Andersson, U., Molina, P. E., Abumrad, N. N., Sama, A. & Tracey, K. J., Science 285, 248-251 (1999).
14. Ombrellino, M., Wang, H., Ajemian, M. S., Talhouk, A., Scher, L. A., Friedman, S. G., Tracey, K. J., Lancet 354, 1446-1447 (1999).
15. Austyn, J. M., Nat Med. 5, 1232-1233 (1999).
16. Gallucci, S. & Matzinger, P., Current Opin. Immunol. 13, 114-119 (2001).
17. Calogero, S., Grassi, F., Aguzzi, A., Voigtlander, T., Ferrier, P., Ferrari, S., Bianchi, M. E., Nat Genet. 22, 276-280 (1999).
18. Ronchetti, A., Rovere, P., Iezzi, G., Galati, G., Heltai, S., Protti, M. P., Garancini, M. P., Manfredi, A. A., Rugarli, C., Bellone M., J. Immunol. 163,130-136 (1999).
19. Albert M L, Sauter B, Bhardwaj N., Nature 392, 86-89 (1998).
20. Inaba K., Turley, S., Yamaide, F., Iyoda, T., Mahnke, K., Inaba, M., Pack, M., Subklewe, M., Sauter, B., Sheff, D., Albert, M., Bhardwaj, N., Mellman, I. & Steinman, R. M., J. Exp. Med. 188, 2163-2173 (1998).
21. Kurts, C., Miller, J. F. A. P., Subramaniam, R. M., Carbone, F. R. & Heath, W. R., J. Exp. Med. 188,409-414 (1998).
22. Steinman, R. M., Turley, S., Mellman, I., Inaba, K., J. Exp. Med. 191, 411-416 (2000).
23. Savill, J. & Fadok, V., Nature 407, 784-788 (2000).
24. Platt, N., da Silva, R. P & Gordon, S., Trends Cell Biol. 8, 365-372 (1998).
25. Gregory, C. D., Current Opin. Immunol. 12, 27-34 (2000).
26. Rovere, P., Peri, G., Fazzini, F., Bottazzi, B., Doni, A., Bondanza, A., Zimmermann, V. S., Garlanda, C., Fascio, U., Sabbadini, M. G., Rugarli, C., Mantovani, A. & Manfredi, A. A., Blood 96, 4300-4306 (2000).
27. Aderem A, Ulevitch R J., Nature 17; 406(6797): 782-7 (August 2000).
28. Ogata H, Su I, Miyake K, Nagai Y, Akashi S, Mecklenbrauker I, Rajewsky K, Kimoto M, Tarakhovsky A., J. Exp Med. (2000) July 3; 192(1): 23-9.
29. Andersson, U., Wang, H., Palmblad, K., Aveberger, A. C., Bloom, O., Erlandsson-Harris, H., Janson, A., Kokkola, R., Zhang, M., Yang, H., and Tracey, K. J. (2000) J. Exp. Med. 192, 565-70.
30. Andrei, C., Dazzi, C., Lotti, L., Torrisi, M. R., Chimini, G., and Rubartelli, A. (1999) Mol. Biol. Cell 10, 1463-75.
31. Andrews, N. W. (2000) Trends Cell Biol. 10, 316-321.
32. Bianchi, M. E., and Beltrame, M. (2000) EMBO Rep. 1, 109-114.
33. Biswas, P., Mantelli, B., Delfanti, F., Cota, M., Vallanti, G., de Filippi, C., Mengozzi, M., Vicenzi, E., Lazzarin, A., and Poli, G. (2001) Cytokine 13, 55-59.
34. Boulikas, T. (1993) Crit. Rev. Eukaryot. Gene Expr. 3, 193-227.
35. Bovolenta, C., Lorini, A. L., Mantelli, B., Camorali, L., Novelli, F., Biswas, P., and Poli, G. (1999) J. Immunol. 162, 323-330.
36. Bustin, M. (1999) Mol. Cell Biol. 19, 5237-46.
37. Calogero, S., Grassi, F., Aguzzi, A., Voigtländer, T., Ferrier, P., Ferrari, S., and Bianchi, M. E. (1999) Nature Genet. 22, 276-280.
38. Cokol, M., Nair, R., and Rost, B. (2001) EMBO Rep. 1, 411-415.
39. Degryse, B., Bonaldi, T., Scaffidi, P., Müller, S., Resnati, M., Sanvito, F., Arrigoni, G., and Bianchi, M. B. (2001) J. Cell Biol. 152, 1197-2006.
40. Falciola, L., Spada, F., Calogero, S., Längst, G., Voit, R., Grummt, I., and Bianchi, M. E. (1997) J. Cell Biol. 137, 19-26.
41. Gardella, S., Andrei, C., Ferrera, D., Lotti, L. V., Torrisi, M. R., Bianchi, M. E., and Rubartelli, A. (2002) EMBO Rep. 3,
42. Hori, O., Yan, S. D., Ogawa, S., Kuwabara, K., Matsumoto, M., Stem, D., and Schmidt, A. M. (1995) J. Biol. Chem. 270, 25752-25761.
43. Kouzarides, T. (2000) EMBO J. 19, 1176-9.
44. Madison, D. L., Yaciuk, P., Kwok R. P. S., Lundblad J. R., J. Biol. Chem., Vol. 277, 41, 38755-38763, Oct. 11, 2002
45. Müller, S., Bianchi, M. E., and Knapp, S. (2001a) Biochemistry 40, 10254-10261.
46. Müller, S., Scaffidi, P., Degryse, B., Bonaldi, T., Ronfani, L., Agresti, A., Beltrame, M., and Bianchi, M. E. (2001b) EMBO J. 20, 4337-4340.
47. Nightingale, K., Dimitrov, S., Reeves, R., and Wolffe, A. P. (1996) EMBO J. 15, 548-561.
48. Scaffidi, P., Misteli, T., and Bianchi, M. E. (2002) Nature 418, 191-195.
49. Scherle, P. A., Jones, E. A., Favata, M. F., Daulerio, A. J., Covington M. B., Nurnberg, S. A., Magolda, R. L., Trzaskos, J. M., J. Immunol., 161: 5681-5686 (1998).
50. Shevchenko, A., Wilm, M., Vorm, O., and Mann, M. (1996) Anal. Chem. 68, 850-8.
51. Soutoglou, E., Katrakili, N., Talianidis, I., Mol. Cell 5, 745-751 (2000).
52. Spilianakis, C., Papamatheakis, J., Kretsovali, A., Mol Cell Biol. 20 (22): 8489-8498 (2000).
53. Sterner, R., Vidali, G., and Allfrey, V. G. (1979) J. Biol. Chem. 254, 11577-11583.
54. Stinchcombe, J. C., and Griffiths, G. M. (1999) J. Cell Biol. 147, 1-6.
55. Sudbeck, P., and Scherer, G. (1997) J. Biol. Chem. 272, 27848-52.
56. Thomas, J. O., and Travers, A. A. (2001) Trends Biochem. Sci. 26, 167-174.
57. Travers, A. A., Ner, S. S., and Churchill, M. E. A. (1994) Cell 77, 167-169.
58. Wang, H., Bloom, O., Zhang, M., Vishnubhakat, J. M., Ombrellino, M., Che, J., Frazier, A., Yang, H., Ivanova, S., Borovikova, L., Manogue, K. R., Faist, E., Abraham, E., Andersson, J., Andersson, U., Molina, P. E., Abumrad, N. N., Sama, A., and Tracey, K. J. (1999a) Science 285, 248-51.
59. Wang, H., Vishnubhakat, J. M., Bloom, O., Zhang, M., Ombrellino, M., Sama, A., and Tracey, K. J. (1999b) Surgery 126, 389-92.
60. White, D. A., Belyaev, N. D., and Turner, B. M. (1999) Methods 19, 417-24.

1-55. (canceled)

56. An isolated multiply acetylated protein HMGB1, or a variant or fragment thereof, or a polynucleotide encoding therefore.

57. The isolated acetylated HMGB1 according to claim 56 with the proviso that lysines 2 and 11 are not acetylated.

58. The isolated acetylated protein according to claim 56 derivable from a myeloid cell.

59. The protein according to claim 56 wherein at least one nuclear localization signal is acetylated.

60. The protein of claim 56 wherein one or more of lysines 27, 28, 29, 179, 181, 182, 183 or 184 are acetylated.

61. The protein of claim 56 having the acetylation pattern of FIG. 2C.

62. An isolated acetylated HMGB1 derivable from a myeloid cell.

63. A protein according to claim 62 in which at least one nuclear localization signal is acetylated.

64. The protein according to claim 62 wherein one or more of lysines 27, 28, 29, 179, 181, 182, 183 or 184 or are acetylated.

65. The protein according to claim 62 having the acetylation pattern of FIG. 2C.

66. A polynucleotide encoding for the protein of claim 56.

67. A pharmaceutical composition comprising the acetylated protein HMGB1 of claim 56 and pharmaceutically acceptable carrier, excipient or diluent.

68. An inhibitor of an acetylated HMGB1 or a fragment thereof, wherein said inhibitor has greater specificity for acetylated HMGB1 over non-acetylated HMGB1.

69. The inhibitor of claim 68, wherein said inhibitor inhibits the acetylation of HMGB1.

70. The inhibitor of claim 68, wherein said inhibitor is selected from the group consisting of a receptor antagonist and an antibody or an antigen-binding fragment thereof.

71. The antibody or antigen-binding fragment of claim 70 wherein said antibody or fragment is polyclonal.

72. The antibody or antigen-binding fragment of claim 70 wherein said antibody or fragment is monoclonal.

73. The antibody or antigen-binding fragment of claim 70 wherein said antibody or fragment is humanized, single-chain or chimeric.

74. The antigen-binding fragment of claim 70 wherein said fragment is selected from the group consisting of a Fab fragment, Fab′ fragment, a F(ab′)₂ fragment and a Fv fragment.

75. The antibody or antigen-binding fragment of claim 70 wherein said antibody or antigen-binding fragment binds to a peptide consisting of the amino acid sequence AKKGVVKAEKSKKKKE wherein each lysine of said peptide is acetylated.

76. A pharmaceutical composition comprising the inhibitor of claim 68 and a pharmaceutically acceptable carrier or excipient.

77. The pharmaceutical composition of claim 76 wherein said inhibitor inhibits the acetylation of HMGB1.

78. The pharmaceutical composition of claim 76 wherein said inhibitor is selected from the group consisting of a receptor antagonist and an antibody or an antigen-binding fragment thereof.

79. The pharmaceutical composition of claim 76 further comprising an antagonist of a cytokine selected from the group consisting of TNF, IL-1a, IL-1B, MIF and IL-6.

80. A method of treating an inflammatory condition comprising administering an effective amount of the inhibitor of claim 68.

81. The method of claim 80, wherein said inhibitor inhibits the acetylation of HMGB1.

82. The method of claim 80, wherein said inhibitor is selected from the group consisting of a receptor antagonist and an antibody or an antigen-binding fragment thereof.

83. The method of claim 80, wherein said inflammatory condition is selected from the group consisting of sepsis, acute pancreatitis, adult respiratory distress syndrome, reperfusion injury, cardiovascular disease, peritonitis, rheumatoid arthritis, osteoarthritis, inflammatory bowel disease, systemic lupus erythematosus, asthma, organ transplant rejection, graft-versus-host-disease, cachexia, cystic fibrosis, psoriasis and multiple sclerosis.

84. The method of claim 83, wherein said inflammatory condition is selected from the group consisting of sepsis, reperfusion injury, rheumatoid arthritis, multiple sclerosis and cardiovascular disease.
